(19) United States
(12) Patent Application Publication
(10) Pub. No.: US 2025/0161895 A1
(43) Pub. Date: May 22, 2025

(54) CONTINUOUS PROCESS FOR VACCINE PRODUCTION

(71) Applicant: GLAXOSMITHKLINE BIOLOGICALS SA, Rixensart (BE)

(72) Inventors: Philippe Raymond JEHOULET, Rixensart (BE); Pablo Miguel MARTIN SOLADANA, Rixensart (BE); Laurent STRODIOT, Rixensart (BE); Ioannis TSOUMPAS, Rixensart (BE)

(73) Assignee: GLAXOSMITHKLINE BIOLOGICALS SA, Rixensart (BE)

(21) Appl. No.: 19/012,167

(22) Filed: Jan. 7, 2025

Related U.S. Application Data
(63) Continuation of application No. PCT/EP2023/069770, filed on Jul. 17, 2023.

(30) Foreign Application Priority Data
Jul. 19, 2022 (EP) 22386047.9

(51) Int. Cl. B01F 33/30; B01F 25/43; B01F 101/00; B01F 101/22

(52) U.S. Cl. CPC B01F 33/30; B01F 25/43; B01F 2101/22; B01F 2101/2202; B01F 2215/0431

(57) ABSTRACT

The present invention relates inter alia to a continuous process for producing an immunogenic composition using a micro-fluidic or milli-fluidic (MF) system and filling one or more vessels with the immunogenic composition.

### CROSS-REFERENCE TO RELATED APPLICATIONS

This application is a continuation under 35 U.S.C. §§ 111 and 365(c) of International Application No. PCT/EP2023/069770, filed on Jul. 17, 2023, which claims the benefit under 35 U.S.C. § 119(b) of European Patent Commission Application No. EP22386047.9, filed on Jul. 19, 2022; the contents of each of the above-identified applications are hereby incorporated by reference in their entireties.

### SEQUENCE LISTING

The instant application contains a Sequence Listing, which has been submitted electronically in XML format and is hereby incorporated by reference in its entirety. Said XML formatted copy, which was created on Jul. 17, 2023, is named “70065WO01-seql-000001.xml” and is 7,764 bytes in size.

### TECHNICAL FIELD

The present invention relates to continuous processes using micro-fluidic or milli-fluidic (MF) systems for producing immunogenic compositions and related aspects.

### BACKGROUND

The various stages of producing immunogenic compositions (such as vaccines) have traditionally been performed in “batch” mode. Batch mode involves a step-by-step procedure for manufacturing products. Once one batch of immunogenic composition is completed, the next batch begins, and so on. To produce one batch, each upstream step of the process must be completed before the next step can commence. Typically, antigens are produced in batches and these batches of antigens are then formulated via subsequent, discrete, steps to produce batches of vaccine that are suitable for administration to patients. However, before administration to patients, the vaccine must be transferred into vials or syringes in a process known as filling. Often each of these stages needs to be conducted in different facilities or manufacturing suites, leading to the requirement to transfer large (commercial scale) batch containers, which is both hazardous and inefficient. Furthermore, this batch mode approach to vaccine formulation results in long delays before vaccines can be released.

Batch processing costs the pharmaceutical manufacturing industry an estimated $50 billion each year due to inefficiencies, losses, contamination and expenses that come along with product recalls (Macher and Nickerson 2006). Furthermore, and as highlighted by the Covid-19 pandemic, there remain significant challenges relating to timely distribution of vaccines and thus there is a need to improve the efficiency of vaccine manufacture.

There is thus a need to improve the time and cost efficiency of vaccine formulation and/or filling.

The formulation of a vaccine is generally dependent upon the specific vaccine in question but may include mixing for example antigen(s), carriers, buffers, isotonicity agents, stabilisers, bacteriostats or cryoprotectants in a particular order at discrete stages of production in a single vessel or in a separate vessel per stage. A batch of formulated vaccine is typically transferred (often to another location) for vial filling and finishing.

### SUMMARY OF THE INVENTION

The present inventors have found that continuous processes using MF systems can be used to produce immunogenic compositions and fill vessels, substantially without interruption. In some embodiments, such continuous processes are more efficient and/or reduce waste compared to batch mode processes. In particular, a relatively small system may be used, compared to the large tanks required for batch mode production. Logistic pressures may be decreased, as tanks do not need to be transferred from formulation location to filling location. All formulation steps may be achieved in a single location, avoiding risks associated with moving large tanks such as risks of contamination and cold chain breakage. The production volume may be adapted flexibly to meet demand (rather than a fixed volume for production as in batch mode). Furthermore, adaptation of the process from laboratory to manufacturing scale may be simplified: either elements of the system can be duplicated in parallel or the total flow rate may be increased.

It may have been expected that a continuous process has drawbacks relative to batch mode production. The composition of the vaccine being continuously produced may have varied during production, even potentially falling outside specification ranges. The antigen(s) included in the vaccine may have incurred damage during processing. However, it was surprisingly found that these issues were not borne out in practice when using the process of the invention.

Reduced antigen recovery was encountered in a specific embodiment in Example 4 below. However, it was found that this issue could be remedied by using one mixer (e.g., micromixer) per antigen inlet.

Advantages of the embodiments of the invention relative to the prior art may be one or more of the following:
I. Faster production
II. Increased adaptation of production volume to market needs
III. Increased production capacity
IV. Footprint reduction
V. Reduced waste
VI. Reduced quantity of workers required
VII. Reduction or elimination of liquid-air interface
VIII. Reduction or elimination of antigen denaturation
IX. Simplified transportation logistics
X. Increased compatibility with process digitalisation
XI. Avoidance of contamination

In one aspect there is provided a continuous process for producing an immunogenic composition using a micro-fluidic or milli-fluidic (MF) system and filling one or more vessels with the immunogenic composition, the process comprising:
a) introducing one or more antigens into the MF system,
b) introducing one or more further constituents into the MF system,
c) mixing the constituents in the MF system, and
d) removing the immunogenic composition from the MF system by filling the one or more vessels with the immunogenic composition.

In a further aspect there is provided a micro-fluidic or milli-fluidic (MF) system for continuously producing an immunogenic composition and filling one or more vessels with the immunogenic composition, said system comprising:
a) at least two inlets,
b) one or more mixers and
c) one or more sterile filters.

In a further aspect there is provided a vessel which has been filled with an immunogenic composition using the process or MF system of the invention.

In a further aspect there is provided an immunogenic composition produced using the process or MF system of the invention.

Further aspects of the invention will be evident from the detailed description below.

### BRIEF DESCRIPTION OF THE SEQUENCES

SEQ ID NO: 1 Polypeptide sequence of Protein D (fragment with MDP tripeptide from NS1)
SEQ ID NO: 2 Polypeptide sequence of PE-PilA (fusion protein without signal peptide)
SEQ ID NO: 3 Polypeptide sequence of UspA2
SEQ ID NO: 4 Polypeptide sequence of truncated gE

### BRIEF DESCRIPTION OF THE FIGURES

FIG. 1 General concept for an MF system to formulate and fill vaccines in a continuous process.

FIG. 2 An example MF system and parameters for continuous production of a shingles vaccine.

FIG. 3 An example MF system and parameters for continuous production of a shingles vaccine intermediate.

FIG. 4 Protein content in fractions collected during continuous laboratory-scale shingles vaccine production.

FIG. 5 HPLC-SEC profiles for final bulk from continuous laboratory-scale shingles vaccine production compared with antigen drug substance stock.

FIG. 6 HPLC-SEC profiles for randomly selected samples from continuous laboratory-scale shingles vaccine production.

FIG. 7 An example MF system for continuous manufacturing-scale shingles vaccine production.

FIG. 8 HPLC-SEC analysis of manufacturing-scale shingles vaccine produced continuously.

FIG. 9 An example illustration of an MF system 1000

FIG. 10 An example illustration of an MF system 2000

FIG. 11 An example illustration of a micromixer 3000

FIG. 12 Schematic diagrams A to H of the micromixer setups used in Example 4

FIG. 13 Ribogreen labelling and dynamic light scattering results for Example 5

FIG. 14 Results of HPLC SEC and Lowry analysis for Example 6

### DETAILED DESCRIPTION OF THE INVENTION

### Immunogenic Compositions and Constituents Thereof

The invention provides a method/process and a system/device for a continuous process for producing an immunogenic composition. The immunogenic composition may be for example a vaccine or a vaccine intermediate.

An “immunogenic composition” is a composition suitable for administration to a subject (e.g. in an experimental or clinical setting) that is capable of eliciting an immune response, i.e. against an antigen. An immunogenic composition may also be a composition suitable for administration to a human and capable of eliciting an immune response if one or more further constituents are added to the immunogenic composition (e.g. water added to a concentrated composition) before administration. Suitably the immunogenic composition is in the form of a liquid, such as a suspension, solution or emulsion. Typically the immunogenic composition is in a single phase.

A vaccine is an immunogenic composition which is suitable for administration (e.g. immediate administration) to a subject. Suitably a vaccine for the purposes of the invention is capable of eliciting an immune response which provides protection against infection by one or more particular pathogens.

A vaccine intermediate is an immunogenic composition which, upon addition of one or more constituents, will be suitable for administration to a subject and to elicit an immune response. It may be desirable to produce a vaccine intermediate using the process/methods and/or system/device of the invention, then store the vaccine intermediate for later use in production of a vaccine (e.g. by dilution of a concentrated vaccine intermediate or rehydration of a lyophilised vaccine intermediate, using water). In one embodiment, the vaccine intermediate is suitable for lyophilisation or is lyophilised. Suitably, the vaccine intermediate is suitable for administration to a subject after dilution or after rehydration.

By an “immunogenic composition constituent” (or “constituent”) is meant a substance which may be present in an immunogenic composition. The substance may contribute to immunogenicity or another property of the composition. Suitably the constituents are in the same phase.

Suitably steps a) to d) are performed in order. Suitably the process of the invention substantially consists of, such as consists of, the recited steps.

### Antigens

An antigen is a constituent. One or more antigens are introduced into the MF system and incorporated into the immunogenic composition according to the process/system of the invention.

By the term antigen is meant a polypeptide, polynucleotide or polysaccharide which is capable of eliciting an immune response. Suitably the antigen comprises at least one B or T cell epitope. The elicited immune response may be an antigen specific B cell response, which produces neutralizing antibodies. The elicited immune response may be an antigen specific T cell response, which may be a systemic and/or a local response. The antigen specific T cell response may comprise a CD4+ T cell response, such as a response involving CD4+ T cells expressing a plurality of cytokines, e.g. IFNgamma, TNFalpha and/or IL2. Alternatively, or additionally, the antigen specific T cell response comprises a CD8+ T cell response, such as a response involving CD8+ T cells expressing a plurality of cytokines, e.g., IFNgamma, TNFalpha and/or IL2. The antigen may be derived (such as obtained from) from a human or non-human pathogen including, e.g., bacteria, fungi, parasitic microorganisms or multicellular parasites which infect human and non-human vertebrates, or from a cancer cell or tumor cell. Most suitably the one or more antigens are polypeptides, such as recombinant polypeptides.

The amount of antigen to be included in the immunogenic composition is suitably an amount which induces an immunoprotective response without significant, adverse side effects. Such amount will vary depending upon which specific immunogen is employed and how it is presented.

One or more antigens are introduced into the MF system. Suitably, one antigen is introduced into the MF system. Alternatively, two or more, such as three or more, such as four or more, such as five or more antigens are introduced into the MF system. In one embodiment, no more than three, such as no more than two antigens are introduced into the MF system. Suitably the one or more antigens are provided in one or more containers. Most suitably, each antigen is provided in a separate container.

An exemplary antigen is an antigen derived from Varicella zoster virus (VZV). The VZV antigen may be any suitable VZV antigen or immunogenic derivative thereof, suitably being a purified VZV antigen. In one embodiment, the VZV antigen is the VZV glycoprotein gE (also known as gp1) or immunogenic derivative hereof. The wild type or full length gE protein consists of 623 amino acids comprising a signal peptide, the main part of the protein, a hydrophobic anchor region (residues 546-558) and a C-terminal tail. In one aspect, a gE C-terminal truncate (also referred to truncated gE or gE truncate) is used whereby the truncation removes 4 to 20 percent of the total amino acid residues at the carboxy terminal end. In a further aspect, the truncated gE lacks the carboxy terminal anchor region (suitably approximately amino acids 547-623 of the wild type sequence). In a further aspect gE is a truncated gE having the sequence of SEQ ID NO. 4. The gE antigen, anchorless derivatives thereof (which are also immunogenic derivatives) and production thereof is described in EP0405867 and references therein (see also Vafai 1994). EP0192902 also describes gE and production thereof. Truncated gE is also described by Haumont et al. 1996. An adjuvanted VZV gE composition suitable for use in accordance of the present invention is described in WO2006/094756, i.e. a carboxyterminal truncated VZV gE.

Another antigen that may be employed in accordance with the present invention is an antigen derived from Haemophilus influenzae. The antigen may be non-typeable Haemophilus influenzae antigen for example selected from: Fimbrin protein (U.S. Pat. No. 5,766,608) and fusions comprising peptides therefrom [e.g. LB1(f) peptide fusions; U.S. Pat. No. 5,843,464 or WO 1999/064067]; OMP26 (WO97/01638); P6 (EP0281673); TbpA and/or TbpB; Hia; Hsf; Hin47; Hif; Hmw1; Hmw2; Hmw3; Hmw4; Hap; D15 (WO1994/012641); Protein D (EP0594610); P2; and P5 (WO1994/026304); protein E (WO2007/084053) and/or PilA (WO2005/063802).

The antigen may be Moraxella catarrhalis protein antigen(s), for example selected from: OMP106 (WO1997/041731 & WO1996/034960); OMP21; LbpA &/or LbpB (WO1998/055606); TbpA &/or TbpB (WO1997/013785 & WO1997/032980); CopB (Helminen et al. 1993); UspA1 and/or UspA2 (WO1993/003761); OmpCD; HasR (WO1999/064602); PilQ (WO1999/064448); OMP85 (WO2000/052042); lipo06 (GB9917977.2); lipo10 (GB9918208.1); lipo11 (GB9918302.2); lipo18 (GB9918038.2); P6 (WO1999/057277); D15 WO1999/063093); OmplA1 (WO2000/015802); Hly3 (WO1999/058684); and OmpE.

In an embodiment, the one or more antigens may be non-typeable H. influenzae (NTHi) protein antigen(s) and/or M. catarrhalis protein antigen(s). Suitably, the antigens may be Protein D (PD) from H. influenzae. Protein D may be as described in WO1991/018926. Suitably Protein D comprises or consists of SEQ ID NO: 1. Alternatively, or in addition, the antigen may be Protein E (PE) and/or Pilin A (PilA) from H. Influenzae. Protein E and Pilin A may be as described in WO2012/139225. Protein E and Pilin A may be presented as a fusion protein; for example LVL735 as described in WO2012/139225. Suitably the PE-PilA fusion protein comprises or consists of SEQ ID NO: 2. For example, three NTHi antigens (PD, PE and PilA, with the two last ones combined as a PE-PilA fusion protein) may be provided. The antigen may be UspA2 from M. catarrhalis. UspA2 may be as described in WO2015/125118, for example MC-009 ((M) (UspA2 31-564) (HH)) described in WO2015/125118. Suitably the UspA2 antigen comprises or consists of SEQ ID NO: 3.

In one embodiment, the antigens are Protein D, PE-PilA and UspA2. Suitably the concentration of Protein D to be included in the immunogenic composition is 5-100 ppm, such as 10-70 ppm, especially 15-50 ppm, in particular 20-25 ppm, such as 21-23 ppm, such as about 22.4 ppm. Suitably the concentration of PE-PilA to be included in the immunogenic composition is 5-100 ppm, such as 10-70 ppm, especially 15-50 ppm, in particular 20-25 ppm, such as 21-23 ppm, such as about 22.4 ppm. Suitably the concentration of UspA2 to be included in the immunogenic composition is 1-15 ppm, such as 3-10 ppm, especially 5-10 ppm, in particular 6-8 ppm, such as about 7.4 ppm.

In aspects, the one or more antigens may be formulated as a solution with one or more further constituents, which is fed into the MF system.

In one embodiment, the antigen is a polypeptide. In one embodiment, the antigen is not RNA, such as not a polynucleotide. In one embodiment, the vaccine produced by the method of the invention does not comprise RNA, such as does not comprise a polynucleotide.

### Further Constituents

An immunogenic composition according to the invention in addition to the one or more antigens also comprises one or more further constituents. Further constituents include, for example, carriers (such as water or saline), buffers, isotonicity agents, stabilisers, bacteriostats and cryoprotectants. Suitably the constituents are liquids. Suitably the liquids may be solutions or mixtures, such as suspensions.

Buffers are aqueous solutions used to resist changes in pH. Examples of buffers include acetate, citrate, histidine, maleate, phosphate, succinate, tartrate and TRIS. In one embodiment, the buffer is a phosphate buffer, such as Na/Na₂PO₄, Na/K₂PO₄ or K/K₂PO₄. Suitably the buffer is present in the immunogenic composition in an amount of at least 6 mM, at least 10 mM, at least 20 mM or at least 40 mM. Suitably the buffer can be present in the immunogenic composition in an amount of less than 100 mM, less than 60 mM or less than 40 mM.

An isotonicity agent is a compound that is physiologically tolerated and imparts a suitable tonicity to a formulation to prevent the net flow of water across cell membranes that are in contact with the formulation. In some embodiments, the isotonicity agent used for the composition is a salt (or mixtures of salts), conveniently the salt is sodium chloride, suitably at a concentration of approximately 150 nM. In other embodiments, however, the composition comprises a non-ionic isotonicity agent and the concentration of sodium chloride in the composition is less than 100 mM, such as less than 80 mM, e.g. less than 50 mM, such as less 40 mM, less than 30 mM and especially less than 20 mM. The ionic strength in the composition may be less than 100 mM, such as less than 80 mM, e.g. less than 50 mM, such as less 40 mM or less than 30 mM.

Stabilisers are substances which help the pharmaceutical composition to maintain its desirable properties (e.g. reduce degradation) until administration. Stabilisers in particular include surfactants, such as polysorbates, poloxamers or methionine. Particular polysorbates include polysorbate 20 (polyoxyethylene (20) sorbitan monolaurate), polysorbate 40 (polyoxyethylene (20) sorbitan monopalmitate), polysorbate 60 (polyoxyethylene (20) sorbitan monostearate) and polysorbate 80 (polyoxyethylene (20) sorbitan monooleate). A particular poloxamer is poloxamer 188.

Bacteriostats are substances which reduce or stop bacteria from multiplying, while not necessarily killing the bacteria.

Cryoprotectants are substances used to protect biological materials from freezing damage (i.e. due to ice crystal formation). Cryoprotectants include polyols, such as sucrose.

The pH of an immunogenic composition may be adjusted in view of the components of the composition and suitability for administration to the subject. Suitably, the pH of the composition is at least 4, at least 5, at least 5.5, at least 5.8, at least 6. The pH of the liquid may be less than 9, less than 8, less than 7.5 or less than 7. In other embodiments, pH of the liquid is between 4 and 9, between 5 and 8, such as between 5.5 and 8. Consequently, the pH will suitably be between 6-9, such as 6.5-8.5. In a particularly preferred embodiment the pH is between 5.8 and 6.4.

It is well known that for parenteral administration, solutions should have a pharmaceutically acceptable osmolality to avoid cell distortion or lysis. A pharmaceutically acceptable osmolality will generally mean that solutions will have an osmolality which is approximately isotonic or mildly hypertonic. Suitably immunogenic compositions for parenteral administration will have an osmolality in the range of 250 to 750 mOsm/kg, for example, the osmolality may be in the range of 250 to 550 mOsm/kg, such as in the range of 280 to 500 mOsm/kg. In a particularly preferred embodiment the osmolality may be in the range of 280 to 310 mOsm/kg.

Osmolality may be measured according to techniques known in the art, such as by the use of a commercially available osmometer, for example the Advanced™ Model 2020 available from Advanced Instruments Inc. (USA).

Typically the constituents will be introduced at a higher concentration than the concentration at which they are present in the immunogenic composition.

In one embodiment, an enzymatic reaction does not take place in the continuous process. In a further embodiment, a reaction does not take place in the continuous process. In a further embodiment, solely mixing and homogenisation takes place in the continuous process.

### Micro-Fluidic or Milli-Fluidic (MF) Systems

A micro-fluidic or milli-fluidic (MF) system is a fluid handing apparatus typically having dimensions on a micrometre (μm) or millimetre (mm) scale and typically mixing occurs through passive means (i.e. through contact of fluid streams and without moving parts within the mixer). In some embodiments, pumps may propel fluids (e.g., water, a buffer containing solution, an antigen containing solution) through the MF system. An ‘MF system’ is used herein to refer to a micro-fluidic or milli-fluidic system.

A micro-fluidic system is a fluid handing apparatus having elongate fluid flow channels of no greater than 999 μm internal diameter. Thus in an embodiment, a micro-fluidic system is a fluid handing apparatus having elongate fluid flow channels of no greater than 999 μm internal diameter, such as no greater than 900 μm internal diameter, such as no greater than 800 μm internal diameter, such as no greater than 700 μm internal diameter, such as no greater than 600 μm internal diameter, such as no greater than 500 μm internal diameter, such as no greater than 400 μm internal diameter, such as no greater than 300 μm internal diameter, such as no greater than 200 μm internal diameter, such as no greater than 100 μm internal diameter. In one embodiment, a micro-fluidic system is a fluid handing apparatus having elongate fluid flow channels of no greater than 10 μm internal diameter, such as no greater than 9 μm internal diameter, such as no greater than 8 μm internal diameter, such as no greater than 7 μm internal diameter, such as no greater than 6 μm internal diameter, such as no greater than 5 μm internal diameter, such as no greater than 4 μm internal diameter, such as no greater than 3 μm internal diameter, such as no greater than 2 μm internal diameter, such as no greater than 1 μm internal diameter.

In one embodiment, a micro-fluidic system is a fluid handing apparatus having elongate fluid flow channels of 1 μm to 999 μm internal diameter, such as 5 μm to 950 μm internal diameter, such as 10 to 900 μm internal diameter, such as 25 to 850 μm internal diameter, such as 50 to 800 μm internal diameter such as 100 to 750 μm internal diameter, such as 150 to 700 μm internal diameter, such as 200 to 650 μm internal diameter, such as 250 to 600 μm internal diameter or such as 275 to 550 μm internal diameter. In one embodiment, a micro-fluidic system is a fluid handing apparatus having elongate fluid flow channels of 1 μm to 10 μm internal diameter, such as 2 μm to 9 μm internal diameter, such as 3 to 8 μm internal diameter, such as 4 to 7 μm internal diameter, such as 5 to 6 μm internal diameter.

A milli-fluidic system is a fluid handing apparatus having elongate fluid flow channels of greater than 1 mm internal diameter In one embodiment, a milli-fluidic system is a fluid handing apparatus having elongate fluid flow channels of no greater than 100 mm internal diameter, such as no greater than 90 mm internal diameter, such as no greater than 80 mm internal diameter, such as no greater than 70 mm internal diameter, such as no greater than 60 mm internal diameter, such as no greater than 50 mm internal diameter, such as no greater than 40 mm internal diameter, such as no greater than 30 mm internal diameter, such as no greater than 20 mm internal diameter, such as no greater than 10 mm internal diameter.

In one embodiment, a milli-fluidic system is a fluid handing apparatus having elongate fluid flow channels of no greater than 10 mm internal diameter, such as no greater than 9 mm internal diameter, such as no greater than 8 mm internal diameter, such as no greater than 7 mm internal diameter, such as no greater than 6 mm internal diameter, such as no greater than 5 mm internal diameter, such as no greater than 4 mm internal diameter, such as no greater than 3 mm internal diameter, such as no greater than 2 mm internal diameter, such as no greater than 1 mm internal diameter.

In one embodiment, a milli-fluidic system is a fluid handing apparatus having elongate fluid flow channels of 1 mm to 100 mm internal diameter, such as 20 mm to 90 mm internal diameter, such as 30 to 80 mm internal diameter, such as 40 to 70 mm internal diameter, such as 50 to 60 mm internal diameter. In one embodiment, a milli-fluidic system is a fluid handing apparatus having elongate fluid flow channels of 1 mm to 10 mm internal diameter, such as 2 mm to 9 mm internal diameter, such as 3 to 8 mm internal diameter, such as 4 to 7 mm internal diameter, such as 5 to 6 mm internal diameter.

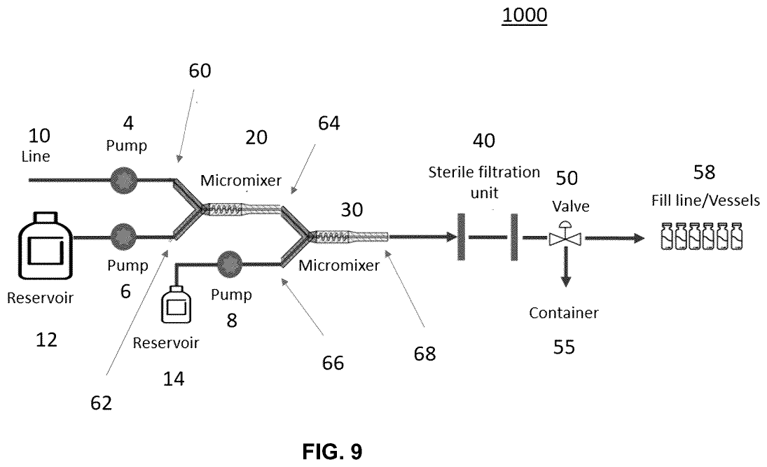

FIG. 9 shows an example illustration of a MF system 1000. A first inlet 60 from micromixer 20 is fluidly connected to line 10 and a second inlet 62 is fluidly connected to reservoir 12. A first pump 4 may be disposed between line 10 and the first inlet 60; a second pump 6 may be disposed between reservoir 12 and the second inlet 62. The fluids mix as the two fluids pass through micromixer 20. The first inlet 64 of micromixer 30 (third inlet in the system) is fluidly connected to the outlet (not labelled) of micromixer 20. The second inlet 66 of micromixer 30 (fourth inlet in the system) is fluidly connected to reservoir 14. A third pump 8 may be disposed between reservoir 14 and the second inlet 66. The fluids mix during passage through micromixer 30. Sterile filter unit 40 is fluidly connected to the outlet 68 of micromixer 30. Value 50 is fluidly connected to the output of the sterile filter unit 40. Value 50 controls flow into a container 55 or filling line 58 (comprising one or more vessels).

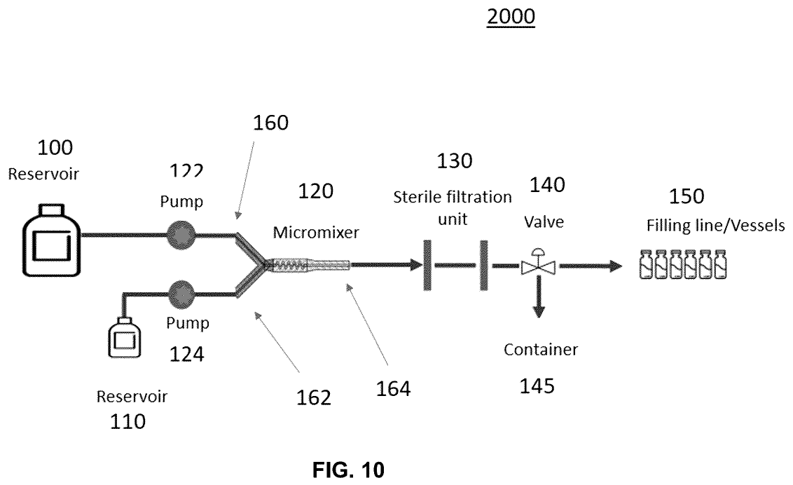

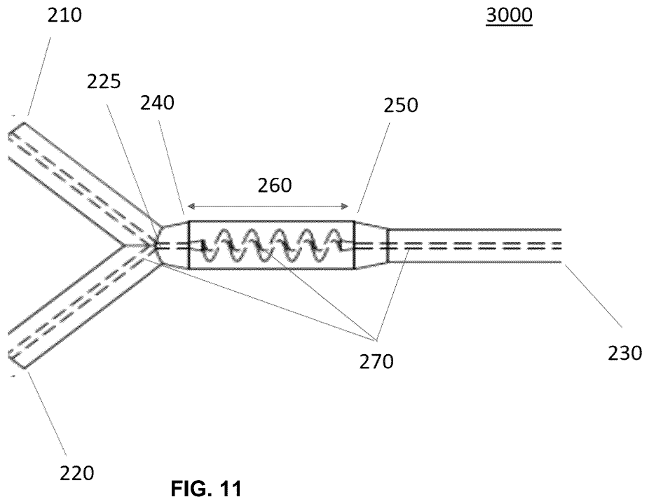

FIG. 10 shows another example illustration of a MF system 2000. A first inlet 160 (see also, FIG. 11) of micromixer 120 receives fluid from reservoir 100 and a second inlet 162 receives fluid from reservoir 110. Thus, reservoir 100 is fluidly connected to inlet 160 and reservoir 110 is fluidly connected to inlet 162. A first pump 122 may be disposed between reservoir 100 and the first inlet 160; a second pump 124 may be disposed between reservoir 110 and the second inlet 162. The two fluids mix during passage through micromixer 120. Sterile filter unit 130 is fluidly connected to the outlet 164 of micromixer 120. Valve 140 is fluidly connected to the output of the sterile filter unit 130. The value 140 controls flow into a container 145 or a filling line 150 (comprising one or more vessels). A reservoir may be a line supply of water, buffer or other solution, or a container with a fixed volume, or any other suitable component for supplying fluid input to the MF system.

FIG. 11 shows an example illustration of a micromixer 3000. A first inlet 210 of micromixer 3000 receives a first fluid, and a second inlet 220 receives a second fluid. The first inlet and the second inlet merge into a single flow path 225 upstream of the channel 260. The first and second fluids mix as the two fluids pass through the channel. In aspects, channel 260 may comprise a serpentine channel. The input 240 and the output 250 of the channel 260 are shown. The first inlet 210 and the second inlet 220 are in fluid communication with the input 240 of the channel; and the output 250 of the channel is in fluid communication with outlet 230 of the micromixer 3000. The fluid flow path 270, which is the path of fluid flow in the micromixer, is shown as dashed lines in the interior of the micromixer.

As used herein, in “fluid communication” means that the component(s) is/are structurally arranged to allow passage of fluid (e.g., a first inlet in fluid communication with a line means that fluid may flow via the line to the first inlet).

As used herein, a “line” may be a continuous supply of a fluid, such as water to the MF system. In some aspects, the line may include tubing for supplying the fluid.

As used herein, an “inlet” refers to the portion of the micromixer that receives fluid input. Inlets may merge prior to feeding fluids into a channel for mixing. As used herein, an “outlet” refers to the portion of the micromixer that provides a mixed fluid.

As used herein, a “fluid” may refer to any suitable liquid, buffer, solution, etc. used in the production of an immunogenic composition.

As used herein, a “reservoir” may be any suitable container for holding a fluid to be provided to the micromixer.

As used herein, a “channel” refers to a portion of the micromixer where mixing occurs (e.g. the serpentine shape). The channel may have an input fluidly connected to the micromixer inlets and an output fluidly connected to the micromixer outlet.

It is understood that the micromixer provides a path for fluid flow from the inlets to the outlet.

To begin operation of the MF system, the operator typically will initially prime the system by commencing continuous introduction of the immunogenic composition constituents, firstly allowing air to escape from the system and latterly allowing waste such as improperly mixed immunogenic composition to be removed (‘waste priming’, for example by a priming valve or by collection in a waste vessel), until the immunogenic composition from the output consists of the desired concentrations of constituents. At this stage the continuous process has begun.

A “continuous” production process is a process in which the product is produced without interruption. In the present context, the term is to be interpreted to refer to the substantially uninterrupted transfer of constituents through the steps of the process. The movement of the constituents in the system is such that the constituents do not substantially remain in one location in the system, or at one step, for a significant or undesired period of time. The process does not require upstream steps to run to completion before downstream steps may commence. When the continuous process is underway, constituents are being introduced into the system at the same time as earlier-introduced constituents are being mixed and at the same time as the completed immunogenic composition is being removed from the system.

The MF system may be formed from any suitable material, namely one which is tolerant of the one or more constituents. Suitable materials include plastic, silicon, glass and stainless steel. Systems may be prepared from such materials by etching, e.g. silicon systems may be prepared by Deep Reactive Ion Etching (DRIE or plasma etching), glass systems may be prepared by wet etching (HF etching) and plastic systems may be prepared by 3D printing. Chosen materials may be subjected to surface treatment to improve the characteristics of the surface.

### Mixers

The MF system may comprise one or more mixers within which the constituents are mixed. Suitably the one or more mixers comprise a mixing channel having a substantially serpentine shape, crossing a single plane multiple times. This way, turbulence is induced in the flow of the liquid by way of the structure of the channel, facilitating mixing.

Suitably the one or more mixers are micromixers. Micromixers are mixers suitable for use in MF systems. Micromixers suitable for use in the present invention may be static (passive) or dynamic (active). Static mixing involves the mixing of components without the application of external forces, i.e. using solely the movement of the components through the MF system to achieve mixing. A pump may propel fluid into an input of the micromixer, but the mixing is still considered passive within the channel. Ideally such mixing will be enhanced by introducing a complex route through the MF system in a mixing chamber. Static mixing approaches include Y- and T-type flow-, multi-laminating-, split-and-recombine-, chaotic-, jet colliding- and recirculation flow-mixers. Dynamic mixing involves the application of external forces, such as stirrers. Dynamic mixing approaches include acoustic fluid shaking (such as using sonicators), ultrasound (such as using ultra-sonicators), electrowetting-based droplet shaking and microstirrers. Most suitably the micromixers are static micromixers.

Suitably the one or more mixers are not dynamic (active) mixers. In one embodiment, the mixers do not comprise moving parts. In one embodiment the mixers are not sonicators.

Desirably the flow rates measured in each mixing chamber vary by less than 5% from the desired flow rate. Suitably the mixers are capable of producing immunogenic composition at a total rate of 50-2000 ml/min, such as 50-1000 ml/min, in particular 100-500 ml/min, for example about 200 ml/min.

The mixing chamber comprised within the mixer should be of adequate length to allow for mixing to be substantially complete by the time liquid reaches the outlet(s). Typically, the chamber will be 1-10 cm in length, such as 1.5-5 cm, especially 1.8-4 cm, in particular 2-3 cm, for example 2.5 cm. Suitably the chamber will be at least 1 cm in length, such as at least 2 cm in length.

In a preferred aspect, the micromixer is Y shaped, with the serpentine path for fluid flow in the body of the Y-shape.

High shear forces within the one or more mixers which may damage the one or more antigens should be avoided. Suitably, the constituents within the one or more mixers incur a shear rate of no greater than 20000 s⁻¹, such as no greater than 2000 s⁻¹, such as no greater than 200 s⁻¹.

Suitably the process does not adversely affect the structure or function of the constituents, such as the one or more antigens.

Ideally the level of mixing in the MF system will be such that a substantially homogenous immunogenic composition is produced, such as a homogenous immunogenic composition.

### Inlets and Outlets

The MF system suitably comprises at least two inlets for introduction of the one or more constituents. Suitably the MF system comprises five or fewer inlets, such as four or fewer inlets, for example three or fewer inlets. Alternatively, the MF system will comprise three or more inlets, such as four or more inlets, for example five or more inlets. To facilitate adequate mixing, the number of inlets for the one or more constituents may be increased for MF systems comprising larger cross-sectional areas, such as comprising mixers with larger cross-sectional areas.

The cross-section of the inlets may be of any shape, though is typically symmetrical. The cross-section may be rectangular (such as square) or circular.

The MF system may comprise at least one valve controlling removal of the immunogenic composition from the MF system by filling the one or more vessels with the immunogenic composition or by diversion into a waste container. The system may have a plurality of valves/outlets for recovery of the immunogenic composition, such as two or three outlets. Most suitably the system will have a single outlet. The cross-section of the outlets may be of any shape, though is typically symmetrical.

It is desirable for the system to achieve a sufficient level of productivity. Additionally, to reduce the impact of startup and shutdown effects it is necessary for the run time to be of adequate length (e.g. at least 30 minutes, especially at least 60 minutes).

The one or more constituents may be stored in separate containers and introduced into the system directly from these separate containers. Alternatively, a plurality of constituents may be combined in a single container (e.g. a concentrated solution comprising buffer and other constituents) for introduction into the MF system.

### Operating Conditions

Optimal operating conditions will depend on the precise configuration of the device and the desired characteristics of the immunogenic composition. Suitably, the flow rate of the final immunogenic composition produced by the process of the invention is 50-2000 ml/min, such as 50-1000 ml/min, in particular 100-500 ml/min, for example about 200 ml/min.

The one or more constituents will typically be provided at a temperature in the region of 10-30° C., such as 15-25° C., in particular 18-22° C. especially 20° C.), and may be at the same or different temperatures, suitably at the same temperature and especially at 20° C. The MF system may be maintained at a temperature in the region of 10-30° C., such as 15-25° C., in particular 18-22° C., especially 20° C. Dependent on the design of the system and environmental conditions it may only be necessary to actively control the temperature of the one or more constituents, and not to actively control the MF system temperature. The MF system may be operated within a controlled temperature environment, e.g. where the temperature is maintained in the range of 10-30° C., such as 15-25° C., in particular about 20° C. (such as 18-22° C., in particular 20° C.).

The operating pressure of the system may be controlled. Suitably, the maximum pressure within the system may be 5 bar, such as 2 bar, such as 1 bar, such as 0.8 bar, such as 0.7 bar, such as 0.6 bar.

Ideally, high shear forces which may damage the one or more antigens should be avoided. High shear may occur anywhere in the MF system but is most likely to occur during mixing. Suitably, the constituents within the MF system incur a shear rate of no greater than 20000 s⁻¹, such as no greater than 2000 s⁻¹, such as no greater than 200 s⁻¹.

### Pumps

The one or more constituents may be introduced into the MF system by any suitable means. Most suitably the constituents are introduced into the MF system by one or more pumps. Typically each constituent is introduced by a separate pump. Suitable pumps include syringe pumps, gear pumps and piston pumps.

It is desirable to use pumps which are adequately precise, substantially pulsation-free and do not significantly degrade the one or more antigens by shear forces. For convenience, it is also desirable for the pumps to be electronically-controlled, such as computer-controlled.

If pumps are used, typically each constituent will be introduced into the MF system by one pump per constituent. The flow rate of the pumps should be adjusted such that the concentration of each constituent is present in the immunogenic composition at a desired concentration. The optimal flow rate will depend on multiple factors including the internal diameter of the tubing, the number of mixers, the number of filters (if any) and the concentration of the constituents. Suitably, the flow rate of the pumps will be set to 1-500 ml/min, such as 5-300 ml/min, in particular 10-200 ml/min.

Suitably, the one or more pumps operate at a pressure of at least 5 bar, such as 2 bar, such as 1 bar, such as 0.8 bar, such as 0.7 bar, such as 0.6 bar.

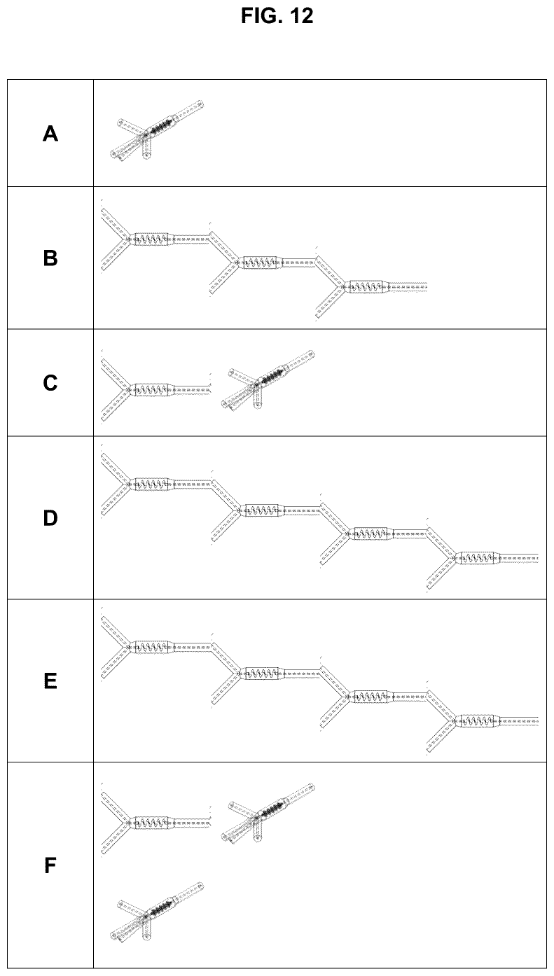

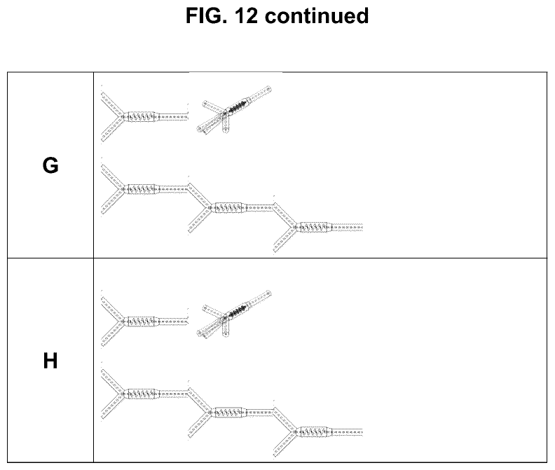

The present inventors have found that if constituents (such as antigens) are introduced into the MF system simultaneously via three or more inlets into a micromixer, this can result in unintentionally reduced quantities of constituents being introduced into the immunogenic composition (see Example 4, wherein the quantity of UspA2 antigen varied in the immunogenic composition being produced). This issue may be overcome by using micromixers with no more than two inlets. If more than two constituents must be introduced in the system, then this issue may be overcome by arranging two-inlet micromixers in series (e.g. substantially as depicted in FIG. 12 in rows D or E of the table therein). By arranging such micromixers in ‘series’ it is meant that the first micromixer receives one constituent per inlet, the outlet of the first micromixer is connected to (and is in fluid communication with) one inlet of the second micromixer, a further constituent is introduced into the second inlet of the second micromixer, and so on. In this way, each micromixer after the first micromixer mixes one constituent with the incoming liquid. By the ‘first’ micromixer is meant the micromixer which receives incoming constituent(s) first in the MF system.

### Sensors and Filters

Suitably one or more sensors are included at the outlet of one or more of the mixers in the MF system. Suitably a sensor is included immediately after the outlet of each mixer in the MF system. The sensors suitably monitor one or more of pH, temperature or pressure. Most suitably the sensors monitor pressure.

Suitably the MF system comprises a filter (e.g. a sterilize filtration unit). For parenteral administration in particular, immunogenic compositions produced by the process of the invention should be sterile. Sterilisation can be performed by various methods although is conveniently undertaken by filtration through a sterile grade filter. By “sterile grade filter” it is meant a filter that produces a sterile effluent after being challenged by microorganisms at a challenge level of greater than or equal to 1×10⁷/cm² of effective filtration area. Sterile grade filters are well known to the person skilled in the art of the invention. For the purpose of the present invention, sterile grade filters have a pore size of 0.1-0.5 μm, suitably 0.18-0.22 μm, such as 0.2 or 0.22 μm. The membranes of the sterile grade filter can be made from any suitable material known to the skilled person, for example, but not limited to cellulose acetate, polyethersulfone (PES), polyvinylidene fluoride (PVDF), polytetrafluoroethylene (PTFE). In a particular embodiment of the invention one or more or all of the filter membranes of the present invention comprise polyethersulfone (PES), in particular hydrophilic polyethersulfone. In a particular embodiment of the invention, the filters used in the processes described herein are a double layer filter, in particular a sterile filter with build-in prefilter having larger pore size than the pore size of the end filter. In one embodiment the sterilizing filter is a double layer filter wherein the pre-filter membrane layer has a pore size between 0.3 and 0.5 nm, such as 0.35 or 0.45 nm. According to further embodiments, filters comprise asymmetric filter membrane(s), such as asymmetric hydrophilic PES filter membrane(s). Alternatively, the sterilizing filter layer may be made of PVDF, e.g. in combination with an asymmetric hydrophilic PES pre-filter membrane layer. In light of the intended medical uses, materials should be of pharmaceutical grade (such as parenteral grade).

If pumps and sensors are utilised in the MF system, then suitably these components may be monitored and controlled automatically or partly automatically using a computer. Suitably the computer is a Process Analytical Technology (PAT) device.

### Vessels and Administration

Suitable vessels include tanks, bags, syringes or vials. If the immunogenic composition is ready for administration to a subject, then suitably the vessels are syringes or vials. If the immunogenic composition requires further processing before administration to a subject, then suitably the vessels are tanks or bags. Most suitably, the vessels are vials.

Suitably the filled vessels produced by the process of the invention are suitable for immediate use in administration to a subject, i.e. transfer to another vessel is not required, other than transfer to a syringe for administration.

Suitably vials may have a volume of 0.5 to 10 ml, such as 1 to 5 ml, such as about 3 ml. Suitably each vial may be filled with 0.1 to 10 ml, such as 0.2 to 5 ml, such as 0.3 to 3 ml of immunogenic composition. Alternatively each vial may be filled with 100-1500 ul, 250-1000 ul, 250-750 ul or 400-600 ul, such as about 500 ul of immunogenic composition.

Suitably the process operates at a filling rate of up to 25 vessels/min, up to 40 vessels/min, up to 60 vessels/min, up to 100 vessels/min, up to 250 vessels/min, up to 400 vessels/min or up to 1000 vessels/min. Suitably the process operates at a filling rate of at least 25 vessels/min, at least 40 vessels/min, at least 60 vessels/min, at least 100 vessels/min, at least 250 vessels/min, at least 400 vessels/min or at least 1000 vessels/min.

The immunogenic composition may be for administration to a subject, such as a mammal, such as a human.

The immunogenic composition may be for administration intradermally, intramuscularly, intraperitoneally or subcutaneously. Suitably the composition is administered intramuscularly.

Sequences
Polypeptide sequence of Protein D
(fragment with MDP tripeptide from NS1)
SEQ ID NO: 1
MDPSSHSSNM ANTQMKSDKI IIAHRGASGY LPEHTLESKA LAFAQQADYL EQDLAMTKDG
RLVVIHDHFL DGLTDVAKKF PHRHRKDGRY YVIDFTLKEI QSLEMTENFE TKDGKQAQVY
PNRFPLWKSH FRIHTFEDEI EFIQGLEKST GKKVGIYPEI KAPWFHHQNG KDIAAETLKV
LKKYGYDKKT DMVYLQTFDF NELKRIKTEL LPQMGMDLKL VQLIAYTDWK ETQEKDPKGY
WVNYNYDWMF KPGAMAEVVK YADGVGPGWY MLVNKEESKP DNIVYTPLVK ELAQYNVEVH
PYTVRKDALP EFFTDVNQMY DALLNKSGAT GVFTDEPDTG VEFLKGIK
Polypeptide sequence of PE-PilA
(fusion protein without signal peptide)
SEQ ID NO: 2
MKYLLPTAAA GLLLLAAQPA MAIQKAEQND VKLAPPTDVR SGYIRLVKNV NYYIDSESIW
VDNQEPQIVH FDAVVNLDKG LYVYPEPKRY ARSVRQYKIL NCANYHLTQV RTDFYDEFWG
QGLRAAPKKQ KKHTLSLTPD TTLYNAAQII CANYGEAFSV DKKGGTKKAA VSELLQASAP
YKADVELCVY STNETTNCTG GKNGIAADIT TAKGYVKSVT TSNGAITVKG DGTLANMEYI
LQATGNAATG VTWTTTCKGT DASLEPANFC GSVTQ
Polypeptide sequence of UspA2
SEQ ID NO: 3
MAKNDITLED LPYLIKKIDQ NELEADIGDI TALEKYLALS QYGNILALEE LNKALEELDE
DVGWNQNDIA NLEDDVETLT KNQNALAEQG EAIKEDLQGL ADFVEGQEGK ILQNETSIKK
NTQRNLVNGF EIEKNKDAIA KNNESIEDLY DFGHEVAESI GEIHAHNEAQ NETLKGLITN
SIENTNNITK NKADIQALEN NVVEELENLS GRLIDQKADI DNNINNIYEL AQQQDQHSSD
IKTLKKNVEE GLLELSGHLI DQKTDIAQNQ ANIQDLATYN ELQDQYAQKQ TEAIDALNKA
SSENTQNIED LAAYNELQDA YAKQQTEAID ALNKASSENT QNIEDLAAYN ELQDAYAKQQ
TEAIDALNKA SSENTQNIAK NQADIANNIN NIYELAQQQD QHSSDIKTLA KASAANTDRI
AKNKADADAS FETLTKNQNT LIEKDKEHDK LITANKTAID ANKASADTKE AATADAITKN
GNAITKNAKS ITDLGTKVDG FDSRVTALDT KVNAFDGRIT ALDSKVENGM AAQAAHH
Polypeptide sequence of truncated gE
SEQ ID NO: 4
SVLRYDDFHI DEDKLDTNSV YEPYYHSDHA ESSWVNRGES SRKAYDHNSP YIWPRNDYDG
FLENAHEHHG VYNQGRGIDS GERLMQPTQM SAQEDLGDDT GIHVIPTLNG DDRHKIVNVN
QRQYGDVFKG DLNPKPQGQR LIEVSVEENH PFTLRAPIQR IYGVRYTETW SELPSLTCTG
DAAPAIQHIC LKHTTCFQDV VVDVDCAENT KEDQLAEISY RFQGKKEADQ PWIVVNTSTL
FDELELDPPE IEPGVLKVLR TEKQYLGVYI WNMRGSDGTS TYATFLVTWK GDEKTRNPTP
AVTPQPRGAE FHMWNYHSHV FSVGDTESLA MHLQYKIHEA PEDLLLEWLY VPIDPTCQPM
RLYSTCLYHP NAPQCLSHMN SGCTFTSPHL AQRVASTVYQ NCEHADNYTA YCLGISHMEP
SFGLILHDGG TTLKFVDTPE SLSGLYVFVV YENGHVEAVA YTVVSYVDHE VNAIEERGEP
PTAGQPPATT KPKEITPVNP GTSPLIRYAA WTGGLA

### Miscellaneous

Throughout the specification, including the claims, where the context permits, the term “comprising” and variants thereof such as “comprises” are to be interpreted as including the stated element (e.g., integer) or elements (e.g., integers) without necessarily excluding any other elements (e.g., integers). Thus a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in or “approximately” in relation to a numerical value x is optional and means, for example, x±10% of the given figure, such as x±5% of the given figure.

As used herein, the singular forms “a,” “an” and “the” include plural references unless the content clearly dictates otherwise.

Unless specifically stated, a process comprising a step of mixing two or more components does not require any specific order of mixing. Thus components can be mixed in any order. Where there are three components then two components can be combined with each other, and then the combination may be combined with the third component, etc.

### Clauses

Clauses further illustrating the invention are as follows:

1. A continuous process for producing an immunogenic composition using a micro-fluidic or milli-fluidic (MF) system and filling one or more vessels with the immunogenic composition, the process comprising:
a) introducing one or more antigens into the MF system,
b) introducing one or more further constituents into the MF system,
c) mixing the constituents in the MF system, and
d) removing the immunogenic composition from the MF system by filling the one or more vessels with the immunogenic composition.

2. The process of clause 1 wherein the immunogenic composition is suitable for lyophilisation.

3. The process of clause 2 wherein the immunogenic composition is lyophilised after filling the one or more vessels.

4. The process of any one of clauses 1 to 3 wherein the immunogenic composition is a vaccine.

5. The process of any one of clauses 1 to 3 wherein the immunogenic composition is a vaccine intermediate.

6. The process of any one of clauses 1 to 5 wherein the immunogenic composition is suitable for administration to a subject.

7. The process of any one of clauses 1 to 5 wherein the immunogenic composition is suitable for administration to a subject after dilution.

8. The process of any one of clauses 1 to 5 wherein the immunogenic composition is suitable for administration to a subject after rehydration.

9. The process of any one of clauses 1 to 8 wherein the immunogenic composition is in the form of a liquid.

10. The process of any one of clauses 1 to 9 wherein the immunogenic composition consists essentially of liquid.

11. The process of clause 10 wherein the immunogenic composition consists of liquid.

12. The process of any one of clauses 11 wherein the immunogenic composition does not comprise solids.

13. The process of any one of clauses 1 to 12 wherein the immunogenic composition does not comprise particles.

14. The process of any one of clauses 1 to 9 wherein the immunogenic composition is a suspension, solution or emulsion.

15. The process of clause 14 wherein the constituents are in the same phase.

16. The process of clause 14 wherein the constituents are in different phases.

17. The process of any one of clauses 1 to 16 wherein the one or more antigens are polypeptides, polynucleotides or polysaccharides.

18. The process of clause 17 wherein the one or more antigens are polypeptides.

19. The process of any one of clauses 1 to 18 wherein only one antigen is introduced into the MF system.

20. The process of any one of clauses 1 to 14 wherein two or more antigens are introduced into the MF system.

21. The process of clause 20 wherein three or more antigens are introduced into the MF system.

22. The process of clause 21 wherein four or more antigens are introduced into the MF system.

23. The process of clause 22 wherein five or more antigens are introduced into the MF system.

24. The process of any one of clauses 1 to 18 wherein no more than three antigens are introduced into the MF system.

25. The process of clause 24 wherein no more than two antigens are introduced into the MF system.

26. The process of any one of clauses 1 to 25 wherein the one or more antigens comprise, such as consist of, an antigen derived from varicella zoster virus (VZV), such as gE.

27. The process of any one of clauses 1 to 26 wherein the one or more antigens comprise, such as consist of, Protein D, PE-PilA and UspA2.

28. The process of any one of clauses 1 to 27 wherein the constituents are liquids.

29. The process of clause 28 wherein the constituents are in the form of a suspension, solution or emulsion.

30. The process of any one of clauses 1 to 29 wherein the constituents are independently selected from carriers, buffers, isotonicity agents, stabilisers, bacteriostats and cryoprotectants.

31. The process of clause 30 wherein the constituents comprise a buffer, a stabiliser, a cryoprotectant and water.

32. The process of either clause 30 or 31 wherein the buffer is selected from acetate, citrate, histidine, maleate, phosphate, succinate, tartrate and TRIS.

33. The process of clause 32 wherein the buffer is a phosphate buffer.

34. The process of clause 33 wherein the phosphate buffer is Na/Na₂PO₄, Na/K₂PO₄ or K/K₂PO₄.

35. The process of any one of clauses 30 to 34 wherein the stabiliser agent is a surfactant, such as a polysorbate.

36. The process of any one of clauses 30 to 35 wherein the cryoprotectant is a polyol, such as sucrose.

37. The process of any one of clauses 1 to 36 wherein each of the constituents are different.

38. The process of any one of clauses 30 to 37 wherein the constituents comprise sucrose, phosphate buffer and polysorbate 80.

39. The process of any one of clauses 1 to 38 wherein the constituents comprise sucrose, phosphate buffer, poloxamer (such as poloxamer 188) and methionine.

40. The process of clauses 1 to 39 wherein mixing is performed with one or more mixers.

41. The process of clause 40 wherein the one or more mixers are micromixers.

42. The process of clause 41 wherein the one or more micromixers are static micromixers.

43. The process of either clause 41 or 42 wherein the one or more micromixers are low shear force micromixers.

44. The process of any one of clauses 41 to 43 wherein the one or more micromixers have a shear force of no greater than 20000 s⁻¹, such as no greater than 2000 s⁻¹, such as no greater than 200 s⁻¹.

45. The process of any one of clauses 1 to 44 wherein the MF system comprises at least two inlets for introduction of the one or more constituents.

46. The process of any one of clauses 1 to 44 wherein the MF system comprises five or fewer inlets, such as four or fewer inlets, for example three or fewer inlets.

47. The process of any one of clauses 1 to 44 wherein the MF system comprises three or more inlets, such as four or more inlets, for example five or more inlets.

48. The process of any one of clauses 1 to 47 wherein each of the constituents are introduced by a different inlet.

49. The process of any one of clauses 1 to 48 wherein the immunogenic composition is produced at a rate of 50-2000 ml/min, such as 50-1000 ml/min, in particular 100-500 ml/min, for example about 200 ml/min.

50. The process of any one of clauses 1 to 49 wherein the constituents within the MF system incur a shear rate of no greater than 20000 s⁻¹, such as no greater than 2000 s⁻¹, such as no greater than 200 s⁻¹.

51. The process of any one of clauses 1 to 50 wherein the constituents are introduced by one or more pumps.

52. The process of clause 51 wherein each constituent is introduced by one pump per constituent.

53. The process of either clause 51 or 52 wherein the pumps provide substantially consistent pressure.

54. The process of any one of clauses 51 to 53 wherein the pumps are substantially pulsation free.

55. The process of any one of clauses 51 to 54 wherein the one or more pumps introduce constituent at a flow rate of 1-500 ml/min, such as 5-300 ml/min, in particular 10-200 ml/min.

56. The process of any one of clauses 1 to 55 wherein the MF system comprises at least one outlet.

57. The process of any one of clauses 1 to 56 wherein the one or more vessels are tanks, bags, syringes or vials.

58. The process of clause 57 wherein the one or more vessels are vials.

59. The process of any one of clauses 1 to 58 wherein the one or more vessels are completely or partially filled.

60. The process of any one of clauses 1 to 59 wherein the constituents are mixed in the MF system after each addition of a constituent and before performing step d).

61. The process of any one of clauses 1 to 60 wherein each constituent is introduced by a different inlet.

62. The process of any one of clauses 1 to 61 wherein each constituent is different.

63. The process of clause 62 wherein different antigens are introduced in each inlet.

64. The process of any one of clauses 1 to 63 wherein the MF system comprises one or more filters, such as one or more sterile grade filters.

65. The process of any one of clauses 1 to 64 wherein the immunogenic composition is filtered, such as sterile filtered, after mixing.

66. The process of clause 65 wherein the immunogenic composition is sterile filtered through a sterile filter comprising a pore size of 0.1-0.5 μm.

67. The process of clause 66 wherein the immunogenic composition is sterile filtered through a sterile filter comprising a pore size of about 0.22 μm.

68. The process of any one of clauses 65 to 67 wherein the immunogenic composition is filtered in the MF system before step d).

69. The process of any one of clauses 1 to 68 wherein characteristics of the immunogenic composition are continuously monitored during the process.

70. The process of clause 69 wherein the monitoring is performed by one or more electronic sensors.

71. The process of either clause 69 or 70 wherein the monitoring is performed on the immunogenic composition which has undergone mixing.

72. The process of any one of clauses 1 to 71 wherein the monitoring is performed on the immunogenic composition before filtering.

73. The process of any one of clauses 1 to 72 wherein the monitoring is performed on the immunogenic composition immediately before step d).

74. The process of any one of clauses 1 to 73 wherein the MF system comprises one or more sensors immediately after the outlet of each mixer.

75. The process of clause 74 wherein the one or more sensors monitor pressure.

76. The process of any one of clauses 70 to 75 wherein the one or more sensors are monitored with a PAT device and the PAT device controls the introduction of the one or more constituents.

77. The process of any one of clauses 1 to 76 wherein a) to d) are performed in order.

78. The process of any one of clauses 1 to 77 substantially consisting of the recited steps.

79. The process of clause 78 consisting of the recited steps.

80. The process of any one of clauses 1 to 79 wherein the constituents are introduced at a higher concentration than the concentration at which they are present in the immunogenic composition.

81. The process of any one of clauses 1 to 80 comprising the step of allowing air to escape from the MF system between step c) and step d).

82. The process of any one of clauses 1 to 81 comprising the step of waste priming between step c) and step d).

83. The process of any one of clauses 1 to 82 wherein no further steps are performed between a) to d).

84. The process of any one of clauses 1 to 83 wherein a substantially homogenous immunogenic composition is produced.

85. The process of any one of clauses 1 to 84 wherein an enzymatic reaction does not take place in the continuous process.

86. The process of any one of clauses 1 to 84 wherein a reaction does not take place in the continuous process.

87. The process of any one of clauses 1 to 84 wherein solely mixing and homogenisation take place in the continuous process.

88. The process of any one of clauses 1 to 87 wherein the process does not adversely affect the structure nor function of the one or more antigens.

89. The process of clause 88 wherein the process does not adversely affect the structure nor function of the constituents.

90. The process of any one of clauses 1 to 89 wherein the process operates at a filling rate of up to 25 vessels/min, up to 40 vessels/min, up to 60 vessels/min, up to 100 vessels/min, up to 250 vessels/min, up to 400 vessels/min or up to 1000 vessels/min.

91. The process of any one of clauses 1 to 90 wherein the process operates at a filling rate of at least 25 vessels/min, at least 40 vessels/min, at least 60 vessels/min, at least 100 vessels/min, at least 250 vessels/min, at least 400 vessels/min or at least 1000 vessels/min.

92. The process of any one of clauses 1 to 91 wherein the one or more vessels are each filled with 100-1500 ul, 250-1000 ul, 250-750 ul or 400-600 ul of immunogenic composition.

93. The process of clause 92 wherein the one or more vessels are each filled with about 500 ul of immunogenic composition.

94. The process or MF system of any one of clauses 1 to 93 wherein less than 0.1 L, such as less than 0.05 L of immunogenic composition is wasted in priming the system before collecting.

95. The process or MF system of any one of clauses 1 to 94 wherein less than 0.1 mL of immunogenic composition is wasted per unit 10 L of immunogenic composition produced.

96. A micro-fluidic or milli-fluidic (MF) system for continuously producing an immunogenic composition and filling one or more vessels with the immunogenic composition, said system comprising:
a) at least two inlets,
b) one or more mixers and
c) one or more sterile filters.

97. The MF system of clause 96 comprising at least three inlets.

98. The MF system of clause 97 comprising at least four inlets.

99. The MF system of clause 98 comprising at least five inlets.

100. The process or MF system of any one of clauses 1 to 99 wherein the one or more mixers are configured in series.

101. A vessel which has been filled with an immunogenic composition using the process or MF system of any one of clauses 1 to 100.

102. An immunogenic composition produced using the process or MF system of any one of clauses 1 to 100.

103. A micro-fluidic or milli-fluidic (MF) system for producing an immunogenic composition comprising:
a line for supplying a first fluid;
a first reservoir for supplying a second fluid;
a first micromixer comprising:
   a first inlet in fluid communication with the line and a second inlet in fluid communication with the first reservoir;
   a first outlet; and
   a first channel for mixing fluids, wherein the first inlet and the second inlet merge upstream of the input to the first channel, wherein the first fluid and the second fluid undergo mixing during passage through the first channel to form a first mixed fluid, and wherein the output of the first channel is in fluid communication with the first outlet; and
a second micromixer comprising:
   a third inlet in fluid communication with the outlet of the first micromixer and a fourth inlet in fluid communication with a reservoir containing a third fluid;
   a second outlet; and
   a second channel for mixing, wherein the third inlet and the fourth inlet merge upstream of the input to the second channel, wherein the third fluid and the fourth fluid undergo mixing during passage through the second channel to form a second mixed fluid, wherein the output of the second channel is in fluid communication with the second outlet.

104. The micro-fluidic or milli-fluidic (MF) system of clause 103 for producing an immunogenic composition further comprising a sterile filtration unit, wherein the input of the sterile filtration unit is in fluid communication with the outlet of the second micromixer.

105. The micro-fluidic or milli-fluidic (MF) system of clause 103 or 104, further comprising a valve, wherein in a first configuration the valve diverts the filtered, second mixed fluid into a container and in a second configuration the valve directs the filtered, second mixed fluid into one or more vessels.

106. The micro-fluidic or milli-fluidic (MF) system of any one of clauses 103 to 105, wherein the first micromixer and the second micromixer each comprise a serpentine shaped channel.

107. The micro-fluidic or milli-fluidic (MF) system of any one of clauses 103 to 106, wherein the line supplies water to the system.

108. The micro-fluidic or milli-fluidic (MF) system of any one of clauses 103 to 107, wherein the first reservoir supplies buffer to the system.

109. The micro-fluidic or milli-fluidic (MF) system of any one of clauses 103 to 108, wherein the second reservoir supplies one or more antigens in a solution to the system.

110. The micro-fluidic or milli-fluidic (MF) system of any one of clauses 103 to 109, wherein the antigen flows through one micromixer.

111. The micro-fluidic or milli-fluidic (MF) system of any one of clauses 103 to 110, wherein the first micromixer and the second micromixer each have only two inlets.

112. The micro-fluidic or milli-fluidic (MF) system of any one of clauses 103 to 111, wherein one or more pumps are present in the system to propel one or more fluids into the one or more micromixers.

113. A method of producing an immunogenic composition using the micro-fluidic or milli-fluidic (MF) system of any one of clauses 103 to 112 comprising:
mixing a first fluid and a second fluid in a first micromixer to produce a first mixed fluid;
mixing the first mixed fluid with a third fluid to produce a second mixed fluid;
filtering the second mixed fluid; and
filling the filtered, second mixed fluid.

114. A micromixer comprising:
a first inlet and a second inlet;
an outlet; and
a channel for mixing, wherein the first inlet and the second inlet merge upstream of the input to the channel, and wherein a first fluid and a second fluid undergo mixing during passage through the channel.

115. The micro-fluidic or milli-fluidic (MF) system of clause 114, wherein the channel is serpentine shaped.

116. The micro-fluidic or milli-fluidic (MF) system of clause 114 or 115, wherein the micromixer has two inlets, with each inlet in fluid communication with the input to the channel.

117. The micro-fluidic or milli-fluidic (MF) system of any one of clauses 114 to 116, wherein the output of the channel is in fluid communication with the outlet of the micromixer.

118. The micro-fluidic or milli-fluidic (MF) system of any one of clauses 114 to 117, wherein a first reservoir supplies buffer to the system and is in fluid communication with the first inlet.

119. The micro-fluidic or milli-fluidic (MF) system of any one of clauses 114 to 118, wherein a second reservoir supplies one or more antigens to the system and is in fluid communication with the second inlet.

120. The micro-fluidic or milli-fluidic (MF) system of any one of clauses 114 to 119, wherein the antigen flows through one micromixer prior to filling.

121. The micro-fluidic or milli-fluidic (MF) system of any one of clauses 114 to 120 further comprising a sterile filtration unit, wherein the input of the sterile filtration unit is in fluid communication with the outlet of the micromixer.

122. The micro-fluidic or milli-fluidic (MF) system of any one of clauses 114 to 121, further comprising a valve, wherein in a first configuration the valve diverts the mixed fluid into a container and in a second configuration the valve directs the mixed fluid into one or more vessels.

123. The micro-fluidic or milli-fluidic (MF) system of any one of clauses 114 to 122, wherein one or more pumps are present in the system to propel one or more fluids into the micromixer.

124. A method of producing an immunogenic composition using the micro-fluidic or milli-fluidic (MF) system of any one of clauses 114 to 123 comprising:
mixing a first fluid and a second fluid in a first micromixer to produce a mixed fluid;
filtering the mixed fluid; and
filling the filtered mixed fluid.

125. The continuous process, micro-fluidic or milli-fluidic (MF) system or method of any one of clauses 1 to 124 wherein the MF system is a fluid handing apparatus having elongate fluid flow channels of no greater than 10 μm internal diameter, such as no greater than 9 μm internal diameter, such as no greater than 8 μm internal diameter, such as no greater than 7 μm internal diameter, such as no greater than 6 μm internal diameter, such as no greater than 5 μm internal diameter, such as no greater than 4 μm internal diameter, such as no greater than 3 μm internal diameter, such as no greater than 2 μm internal diameter, such as no greater than 1 μm internal diameter.

126. The continuous process, micro-fluidic or milli-fluidic (MF) system or method of any one of clauses 1 to 124 wherein the MF system is a fluid handing apparatus having elongate fluid flow channels of 1 μm to 10 μm internal diameter, such as 2 μm to 9 μm internal diameter, such as 3 to 8 μm internal diameter, such as 4 to 7 μm internal diameter, such as 5 to 6 μm internal diameter.

127. The continuous process, micro-fluidic or milli-fluidic (MF) system or method of any one of clauses 1 to 124 wherein the MF system is a fluid handing apparatus having elongate fluid flow channels of no greater than 10 mm internal diameter, such as no greater than 9 mm internal diameter, such as no greater than 8 mm internal diameter, such as no greater than 7 mm internal diameter, such as no greater than 6 mm internal diameter, such as no greater than 5 mm internal diameter, such as no greater than 4 mm internal diameter, such as no greater than 3 mm internal diameter, such as no greater than 2 mm internal diameter, such as no greater than 1 mm internal diameter.

128. The continuous process, micro-fluidic or milli-fluidic (MF) system or method of any one of clauses 1 to 124 wherein the MF system is a fluid handing apparatus having elongate fluid flow channels of 1 mm to 10 mm internal diameter, such as 2 mm to 9 mm internal diameter, such as 3 to 8 mm internal diameter, such as 4 to 7 mm internal diameter, such as 5 to 6 mm internal diameter.

### EXAMPLES

In the examples below, continuous formulation processes were carried out using milli-fluidic systems having elongate fluid flow channels of 4.8 mm internal diameter.

### Example 1: Design of a Continuous Process for Vaccine Production

### Apparatus and Procedure for General Concept

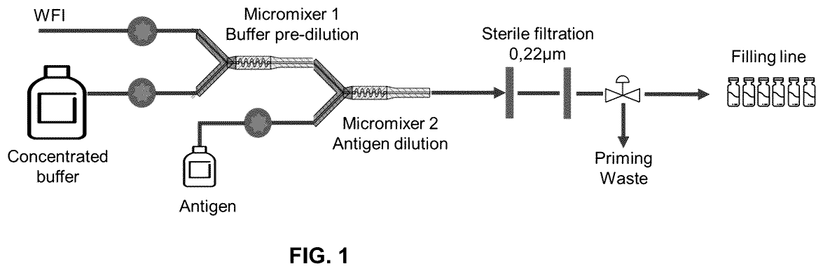

The general concept of an MF system to formulate and fill vaccines in a continuous process was designed (FIG. 1). The MF system was designed to combine water for injection (WFI), concentrated buffer (a concentrated solution comprising buffer and potentially other components) and antigen. The protocol was as follows:

Step 1: This first step is to pre-dilute using a first static micromixer, a concentrated buffer containing all excipients and buffer needed to achieve the formulation with water for injection. In classical batch mode, buffer, water and excipients are prepared separately and added in a determined sequence: water, buffer and excipients.

Step 2: Diluted buffer coming out of step 1 is connected to a second static micromixer where concentrated antigen is added through the other inlet.

Step 3: The formulation coming out of step 2 is complete and can go through the sterile filtration composed of two filters in series: the first one to reduce bioburden and a second one for sterilisation. A 3-way valve is connected on the system to eliminate product during the priming phase and switched to collection when the system has reached a steady state.

Step 4: Filling can be connected through an intermediate bag/tank with a minimal volume enabling to stop or reduce speed of formulation and filling in case of issue.

Starting procedure will be:
1) Start pumping water to purge air from the system, 3-way valve connected to waste.
2) Start pumping concentrated buffer.
3) Increase flow rate to target.
4) Start pumping antigen to target flow rate.
5) After a defined volume/time switch valve to collection.

### Pumps

In this evaluation different pump systems were used depending on scale and production volume:

Cetoni Nemesys syringe pump for lab scale experiments: (connected with glass or stainless steel syringe) (Mid Pressure Syringe Pump neMESYS 1000N|CETONI GmbH)

HNP mikrosysteme: micro annular gear pump hermetic inert pump series (mzr-6355 & 7255) integrated in a Modos System (Filter/pump/Coriolis mass flow controller/Controller). Compatible with continuous processes and GMP requirements, used for lab or manufacturing scale experiments.

Teledyne ISCO pump: dual piston pump for continuous delivery (1000D). Pump only used for manufacturing scale experiment at large volume. This pump system is not compatible with a GMP environment.

### Mixers

Several static micromixer geometries were tested. Micromixers were produced using a 3D printer. Early designs were produced in transparent plastic while USP Class VI transparent resin was used for later designs. Stainless steel micromixers were also produced using a 3D printer.

### Shingles Vaccine

The vaccine produced in these experiments was a vaccine which protects against shingles (herpes zoster) and post-herpetic neuralgia. The vaccine contains glycoprotein E from varicella zoster virus (causing chickenpox). Formulation of the vaccine using classical batch mode is described below. After formulation and sterile filtration, a filling of 0.5 ml per vial before lyophilisation is realized. The lyophilized product is reconstituted with 625 ul of AS01B to reach a concentration of 100 ug/ml of gE antigen.

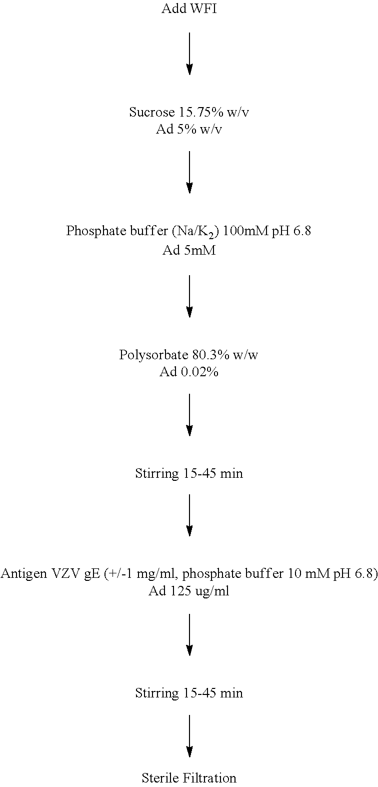

The production of similar vaccines by classical batch mode results in significant waste. Typically around 0.5 L of immunogenic composition is lost in the formulation process and typically around 7 L of immunogenic composition is lost in the filling process. The material used has a value of around 26,000 EUR/L. Reduction of wastage by continuous processing would therefore be highly advantageous.

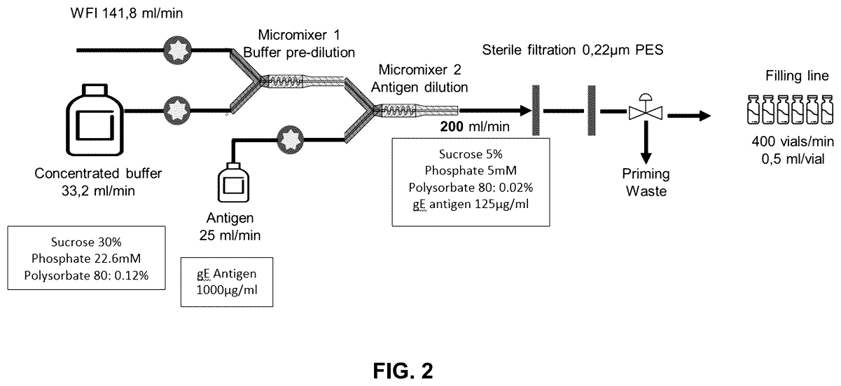

FIG. 2 details a suitable MF system and parameters for continuous production of this shingles vaccine.

For lab scale experiments, targeted total flow rate was 12.5 ml/min and input flow rates were adapted as follows. Concentrations of buffer and antigen remain the same.
Water: 8.86 ml/min
Concentrated buffer: 2.075 ml/min.
Antigen gE: 1.56 ml/min

The concentrated buffer consisted of 30% sucrose, 22.6 mM NaH₂PO₄/K₂HPO₄, 0.12% Polysorbate 80, pH 6.8 when diluted 6×.

### Filtration

Lab scale experiment: Durapore optiscale PVDF 3.5 cm² 0.22 μm.

Reference: SVGLA25NB6 MerckMillipore

Manufacturing scale experiment: Millipak 20 capsule Durapore 0.22 μm

Reference: SIM142A7 MerckMillipore

### Filing and Lyophilisation

Filling volume for the vaccine formulation is 0.5 ml per vial (3 ml siliconized glass vial). After lyophilisation, each vial is reconstituted with 625 ul of AS01B or AS01B buffer for analytics.

### Analytics

gE aggregation was assessed by SEC-HPLC, potency was assessed by ELISA and protein content was assessed by the Lowry method.

H. influenzae/M. catarrhalis Vaccine

A further vaccine produced in these experiments is an H. influenzae/M. catarrhalis vaccine. This vaccine is an AS01-adjuvanted vaccine containing surface proteins from the two main pathogens associated with this disease. The vaccine is trivalent (containing Protein D, PE-PilA & UspA2).

The batch mode formulation process is outlined as follows. Further details on this formulation process are available in WO2021/023691 (see, in particular, Example 1), and the dilution of Protein D in WO2021/023692.

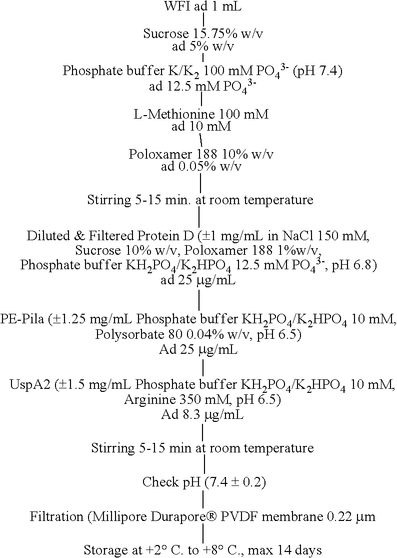

The raw materials were as follows.
Protein D: concentration: 45263 μg/ml
Buffer: 150 mM NaCl
PE-PilA: concentration: 1326 μg/ml
Buffer: 10 mM KH₂PO₄/K₂HPO₄ pH 6.5/Poloxamer 188 0.2% w/v
Uspa2: concentration: 1579 ug/ml
Buffer: 10 mM KH₂PO₄/K₂HPO₄—350 mM Arginine—pH 6.5
Concentrated buffer: 30% Sucrose, 75 mM KH2PO4/K2HPO4, 1.5% Poloxamer 188, 60 mM
Methionine, pH 7.4 when diluted 6×

### Filtration

Only a laboratory-scale experiment was performed, with formulation volumes of 40-60 ml being produced. Manual filtration was performed using a 50 ml plastic syringe with Millex GV PTFE 0.22 μm (33 mm diameter) filters.

### Filling and Lyophilisation

Filling volume for the vaccine is 0.56 ml per vial (3 ml siliconized glass vial). After lyophilisation, vial is reconstituted with 625 ul of AS01B or AS01B buffer for analytics.

### Analytics

Antigen aggregation was assessed by SEC-HPLC. Protein content was assessed by RP-UPLC.

### CFD Analysis

CFD is a numerical analysis tool applied to fluid mechanics. The basis of CFD simulations is the resolution of Navier-Stokes equations (NS), describing the flow of fluids in the domain of interest (i.e. geometry). The term “Mesh” means dividing complex geometry into discrete and simple geometric elements. Meshes are used to perform computational fluid dynamics because these calculations are easier when performed in known geometries, such as triangle. Each analysis performed in triangles is combined (considered interactions between triangles) to get a complete picture. In this study simulations were carried out with Ansys Fluent 19.2 software that uses a control finite-volume-based technique.

### Example 2: Continuous Laboratory-Scale Shingles Vaccine Production

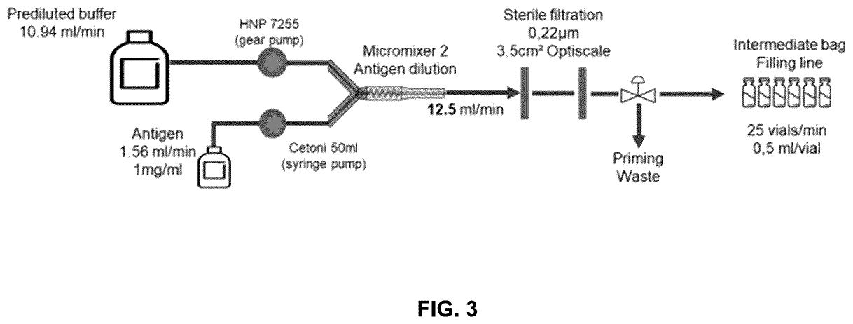

A first experiment was performed at laboratory scale with an MF system where formulation, filtration, filling and lyophilisation were performed at the same location. For this experiment the setup shown in FIG. 3 was used. Masterflex LS15 tubing was used to connect micromixer and filtration systems. A waste bag, an intermediate bag and pressure sensors were also connected.

A syringe pump with a 50 ml stainless steel syringe was used for the antigen line. This limited the formulation to a maximum of 400 ml, but this was enough to perform analytics. Manually prediluted buffer was pumped into the system using an HNP pump. The following procedure was carried out:
1. Buffer pump started with last system valve opened to waste and filter vent opened for air removal. A total volume of 20 ml was needed to completely fill and remove air from the system.
2. Antigen pump started synchronously with buffer pump and fractions of 2 ml were collected at waste exit with a total of 20 fractions.
3. Valve was then switched to intermediate bag and filling line. The intermediate bag was filled with 50-70 ml before starting the filling line.
4. A total of 200 doses of shingles vaccine at 0.5 ml per dose were filled and later lyophilised.

The analysis of the pressure data did not indicate any filter clogging and a maximum of 1 bar was observed for sensor 1 (before first filter) after 1 minute. Protein content analysis was performed using the Lowry method on the waste collected fractions, the starting purified bulk (remaining antigen solution from neMESYS syringe), final bulk from intermediate bag and lyophilised doses (final vessel).

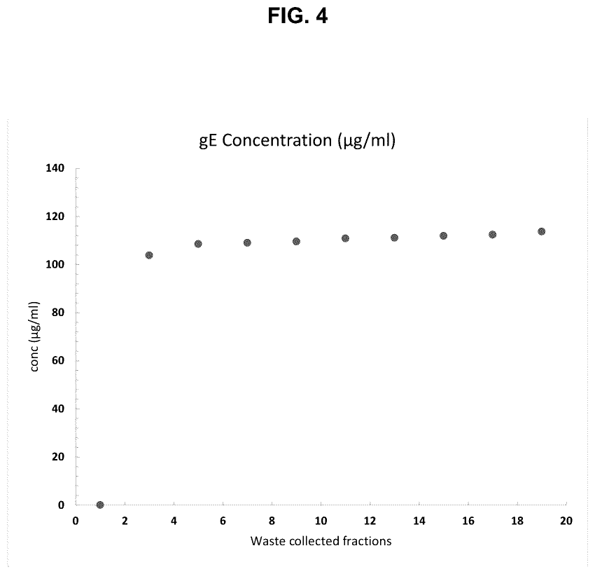

FIG. 4 shows the protein content for all collected fractions. It can be seen that antigen concentration increases rapidly and reaches a steady state after 15-20 ml of collection. This volume could be set as waste volume to be discarded before collection formulation for filling. It is also observed that measured concentrations are lower (110 ug/ml) compared to target (125 ug/ml) but aligned with final bulk concentration of 109 ug/ml (see Table 1). Final vessels after lyophilisation are also lower than expected (target of 100 ug/ml). Purified bulk is close to target suggesting some loss during the formulation process. After investigation, it was noted that a bias could be observed for final bulk and final vessels probably due to the Lowry method and the dilution factor.

TABLE 1

| | Concentration by | Theoretical value | Recovery |
|---|---|---|---|
| Group name | Lowry (ug/ml) | (ug/ml) | (%) |
| Purified bulk | 1014 | 1000 | 101 |
| Final bulk | 109 | 125 | 87 |
| Final container | 89 | 100 | 89 |
| (n = 4) | (+/−0.4) | | |

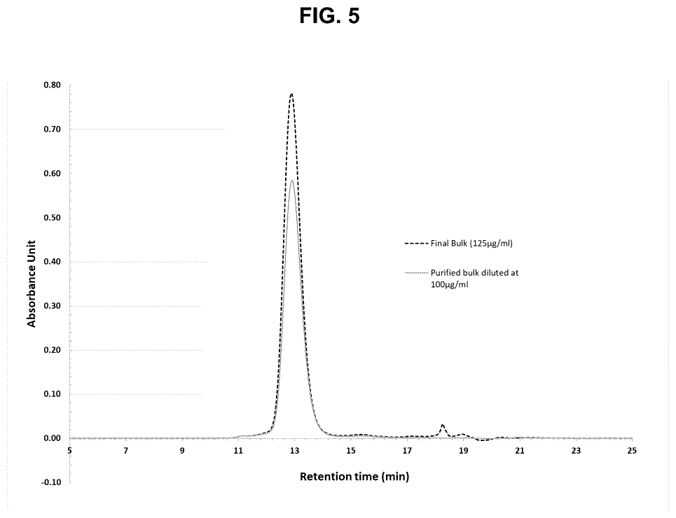

HPLC-SEC analysis was also performed on the different samples to determine if the process impacted the antigen. FIG. 5 shows the profiles for the final bulk (FB, before lyophilisation) compared with the antigen drug substance stock diluted at 100 ug/ml. As expected, peak height is lower for purified pulk (PB) due to the lower concentration, but no aggregation is visible (start of peaks are similar).

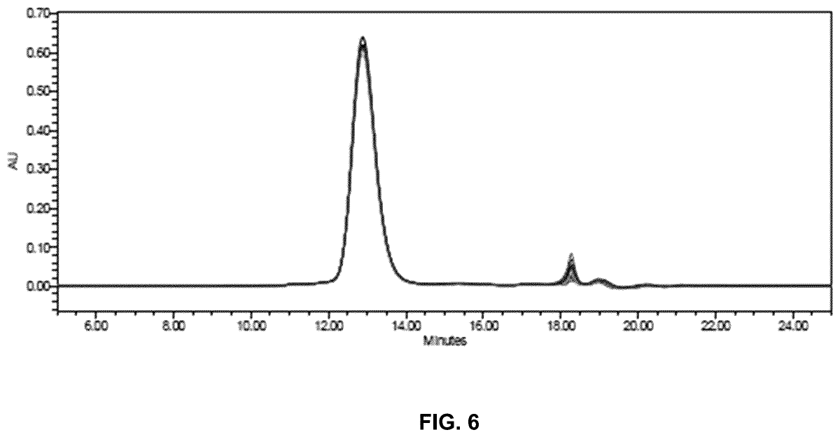

Ten lyophilised samples were randomly selected and analysed (FIG. 6). All chromatograms were comparable with no visible aggregation. A key is not provided because all curves are almost completely overlapping.

Peak areas from lyophilised samples were also compared with diluted purified bulk at 100 ug/ml in order to confirm the Lowry results and the potential antigen loss. Table 2 shows recoveries. It was confirmed that there was no loss, as all samples were in a range of 100 to 108% of recovery.

TABLE 2

| | Average Area | Recovery |
|---|---|---|
| | (HPLC SEC) | (%) |
| Diluted PB (fresh) | 26159938 | |
| (100 ug/ml) | | |
| Final bulk T0 | 34182557 | 104.5 |
| (125 ug/ml) | | |
| Lyo 1 | 26805328 | 102.5 |
| Lyo 2 | 27018704 | 103.3 |
| Lyo 3 | 27045692 | 103.4 |
| Lyo 4 | 26981612 | 103.1 |
| Lyo 5 | 26951288 | 103.0 |
| Lyo 6 | 26271221 | 100.4 |
| Lyo 7 | 26825761 | 102.5 |
| Lyo 8 | 27010308 | 103.3 |
| Lyo 9 | 26190664 | 100.1 |
| Lyo 10 | 28215557 | 107.9 |

Potency tests by ELISA were also performed on 5 samples and it was confirmed that no degradation of the gE antigen had occurred (Table 3).

TABLE 3

| Group | Ratio |
|---|---|
| Lyo 1 | 1.18 |
| Lyo 2 | 1.18 |
| Lyo 3 | 1.14 |
| Lyo 4 | 1.2 |
| Lyo 5 | 1.19 |

In summary, no impact from the continuous production process was apparent from the assessments carried out above.

### Example 3: Continuous Manufacturing-Scale Shingles Vaccine Production

A manufacturing-scale MF system experiment was set up with a total flow rate of 200 ml/min, combining all steps as in FIG. 2: online dilution of concentrated buffer (as defined in Example 2), antigen addition, sterile filtration and filling. The experiment was divided across two locations: the process until filtration was performed at one location and filling was performed at a second location.

Experimental setup was:

Pump systems: ISCO1000D for water, HNP mzr6355 for concentrated buffer and concentrated antigen.

Micromixer: one 3D printed piece containing 2 micromixers connected in series.

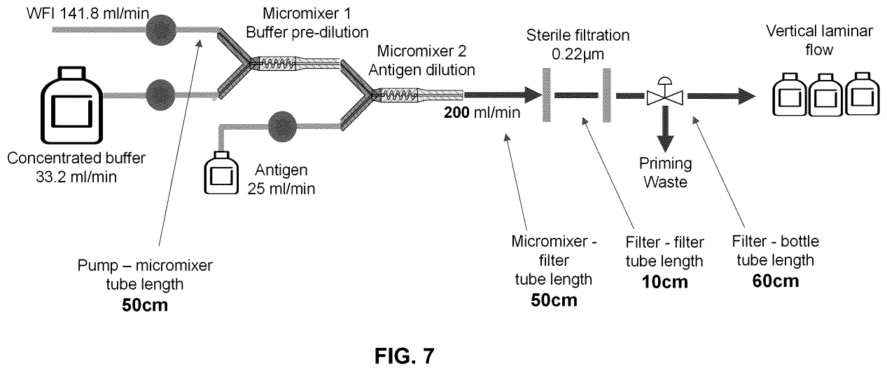

The setup is illustrated in FIG. 7. The following procedure was carried out:
1. Pump primed
2. Flow rate set point procedure: flow rate increased by reaching intermediate steps (see Table 4), next flow rates set when pump flow rate remained stable for 10 seconds. At the same time the filters' vent valves are closed when no more air was present in the filter capsules.
3. When the system reached target flow rate, a waste volume was collected (100 ml) to ensure steady state of the system (stable antigen concentration).

TABLE 4

| Water | Buffer | Antigen |
|---|---|---|
| (ml/min) | (ml/min) | (ml/min) |
| 25 | 0 | 0 |
| 50 | 10 | 0 |
| 100 | 20 | 0 |
| 100 | 23 | 10 |
| 120 | 28 | 15 |
| 141.8 | 33.2 | 25 |

4. After waste collection, exit tubing was connected to a first 1 L bottle and filled at ˜700 ml (named “beginning”), then a second 1 L bottle was filled with ˜700 ml (named “middle”). A third 1 L bottle was filled until the process was stopped (named “End”). Estimated run time was 11 minutes after targeted flow rates were reached to fill all bottles.
5. The contents of the three bottles were used to fill smaller vessels and lyophilized, in order to evaluate the homogeneity of the distribution throughout the run.

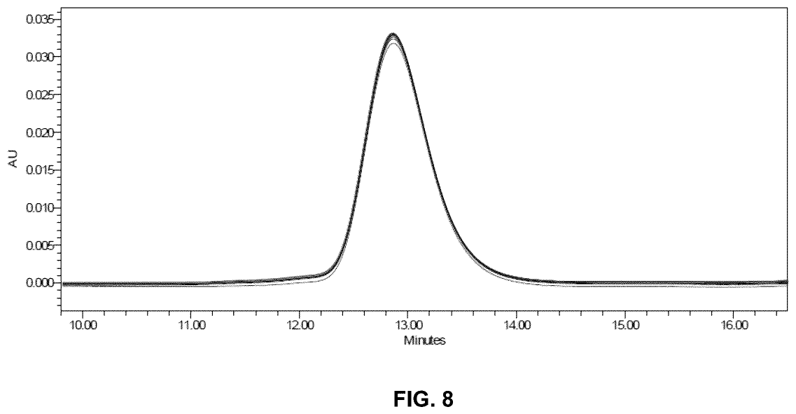

As for the lab-scale experiment above, UV280 nm HPLC-SEC analysis was performed on these 3 samples to determine if the process at high flow rate impacted the antigen. The results are provided in FIG. 8. This figure presents the SEC-HPLC profiles for all three samples with replicates: all profiles were similar and did not show any aggregation. A key is not provided because all curves are almost completely overlapping.

Potency analysis by ELISA was also performed on the three samples and confirmed no degradation of the gE antigen through the process. Table 5 shows the results (specification reference: 0.70-1.30).

TABLE 5

| | Ratio | Ratio |
|---|---|---|
| Group | Test 1 | Test 2 |
| Beginning | 1.06 | 1.06 |
| Middle | 1.01 | 0.97 |
| End | 1.13 | 1.00 |

### Example 4: Continuous Laboratory-Scale H. influenzae/M. Catarrhalis Vaccine Production

An experiment was performed at laboratory scale with an MF system in which an H. influenzae/M. catarrhalis vaccine was prepared continuously. This experiment was carried out to assess if it was possible to continuously produce a vaccine comprising multiple antigens and how this may be best achieved.

Table 6 presents the conditions tested and micromixer configuration.

Groups 1 & 2 compared the use of one or multiple micromixers.

Groups 3 & 4 combined the use of the concentrated buffer and single or multiple micromixers for antigens.

Group 5 compared to group 4 the impact on antigen while passing to micromixers connected in series.

Groups 6 to 8 combined the above comparison and the use of separate excipients (buffer, sucrose, tween 80).

Groups 9 and 10 are controls, formulated classically in single batches, under a laminar flow (to avoid contamination) and assessing the use of a concentrated buffer instead of separate excipients.

TABLE 6

| | | MF system micromixer | Antigens |
|---|---|---|---|
| Group | Procedure | arrangement | addition order |
| 1 | All in one buffer manually diluted. 1 | FIG. 12 in row A | At the same |
| | Micromixer with 4 inlets (1 for the | 1 micromixer (4 inlets) | time |
| | buffer and 3 for each Ag added at the | | |
| | same time in the micromixer). | | |
| 2 | All in one buffer manually diluted. 3 | FIG. 12 in row B | PD - PePilA- |
| | micromixers with 2 inlets, 1 per | 3 micromixers (2 inlets) | UspA2 |
| | antigen | | |
| 3 | All in one buffer concentrate diluted in | FIG. 12 in row C | At the same |
| | one micromixer with 2 inlets and all | 1 micromixer (2 inlets) + | time |
| | antigens added at once in one | 1 micromixer (4 inlets) | |
| | micromixer with 4 inlets | | |
| 4 | All in one buffer concentrate diluted in | FIG. 12 in row D | PD - PePilA- |
| | one micromixer with 2 inlets and 3 | 4 micromixers (2 inlets) | UspA2 |
| | micromixers (2 inlets), one for each | | |
| | antigen | | |
| 5 | All in one buffer concentrate diluted in | FIG. 12 in row E | PePilA-UspA2- |
| | one micromixer with 2 inlets and 3 | 4 micromixers (2 inlets) | PD |
| | micromixers (2 inlets), one for each | | |
| | antigen - but the order of antigens | | |
| | addition is changed (PD at the end) | | |
| 6 | Separate concentrated phosphate | FIG. 12 in row F | At the same |
| | buffer diluted in 1 micromixer (2 | 1 micromixer (2 inlets) + | time |
| | inlets) then the rest of the excipients | 2 micromixers (4 inlets) | |
| | are added at once in 1 micromixer (4 | | |
| | inlets) and the antigens are added at | | |
| | once in 1 last micromixer (4 inlets) | | |
| 7 | Separate concentrated phosphate | FIG. 12 in row G | PD - PePilA- |
| | buffer diluted in 1 micromixer (2 | 1 micromixer (2 inlets) + | UspA2 |
| | inlets) then the rest of the excipients | 1 micromixer (4 inlets) + | |
| | are added at once in 1 micromixer (4 | 3 micromixers (2 inlets) | |
| | inlets) and the antigens are added | | |
| | sequentially in 3 micromixers (2 | | |
| | inlets) | | |
| 8 | Separate concentrated phosphate | FIG. 12 in row H | PePilA-UspA2- |
| | buffer diluted in 1 micromixer (2 | 1 micromixer (2 inlets) + | PD |
| | inlets) then the rest of the excipients | 1 micromixer (4 inlets) + | |
| | are added at once in 1 micromixer (4 | 3 micromixers (2 inlets) | |
| | inlets) and the antigens are added | | |
| | sequentially in 3 micromixers (2 | | |
| | inlets) - but the order of antigens | | |
| | addition is changed (PD at the end) | | |
| 9 | Batch formulation done with separate | NA | PD - PePilA- |
| Control | excipients & buffers | | UspA2 |
| 10 | Batch formulation done with one | NA | PD - PePiIA- |
| Control | buffer containing all excipients - to | | UspA2 |
| | mimic the continuous formulation | | |
| | done with the same buffer (all in one | | |
| | buffer) | | |

FIG. 12, in rows A to H in the table therein, shows schematics of the micromixer setups for the groups above (note that the micromixers for these experiments were connected together, whereas in the schematics they are shown disconnected). For example, FIG. 12, in row D therein, shows the micromixer setup and connections for group 4: the outlet of each micromixer was connected to one inlet of the further micromixer but for the final micromixer. The micromixers were arranged in ‘series’.

The composition of the ‘all in one buffer’ was: 30% w/v Sucrose, 75 mM Phosphate, 0.15% w/v Poloxamer 188, 60 mM Methionine pH 7.4, when diluted 6×. However, the buffer was diluted 6.3× (instead of 6× as per usual for the classical batch formulation) to take into account excipients coming from the antigens' buffers.

The process was as follows:
1. Run of 3 minutes for continuous process at 20 ml/min total flow rate using syringe systems
2. Syringes were completely filled for each group, priming to remove air from all tubing.
3. The water pump was started first, then buffer pump, then excipient pump, then antigen pump (waiting for air bubbles to escape before each pump was started).
4. Last air bubbles were removed, then after 20 seconds, approximately ˜7 ml of liquid was removed for priming and stabilization before starting sample collection.
5. 40-50 ml of vaccine was prepared and collected over 2-3 minutes, using manual sterile filtration.
6. For groups 9 and 10, 50 ml of vaccine was prepared for each group (using classical formulation under laminar flow).
7. 3 ml vials (0.56 ml) were manually filled and underwent lyophilisation cycles (48h).

Table 7 provides the adapted flow rate for each excipient and the antigens used for the continuous processes. After lyophilisation, samples were analysed by RV-HPLC for content analysis (lyophilised cake reconstituted with 625 ul of WFI).

TABLE 7

| | | Buffer | | | | | | | | Exit |
|---|---|---|---|---|---|---|---|---|---|---|
| | | concentrate | | | Poloxamer | | | PE- | | flow |
| | | (X fold | Sucrose | Phosphate | 188 | Methionine | Protein D | Pila | UspA2 | rate |
| | WFI | dilution) | (% w/v) | (mM) | (% w/v) | (mM) | (mg/ml) | (mg/ml) | (mg/ml) | (ml/min) |
| Initial | | 6 | 15.75 | 100 | 10 | 100 | 1 | 1.326 | 1.579 | |
| Target | | 1 | 5 | 12.5 | 0.05 | 10 | 25 | 25 | 8.3 | 20 |
| Dilution | | 6.3 | 3.15 | 8 | 200 | 10 | 40 | 53.04 | 190.2 | |
| factor | | | | | | | | | | |
| Flow | ml/min | ml/min | ml/min | ml/min | ml/min | ml/min | ml/min | ml/min | ml/min | |
| rate per | | | | | | | | | | |
| component | | | | | | | | | | |
| per group | | | | | | | | | | |
| Group 1 | 15.8 | 3.17 | N/A | N/A | N/A | N/A | 0.50 | 0.38 | 0.105 | 20 |
| Group 2 | 15.8 | 3.17 | N/A | N/A | N/A | N/A | 0.50 | 0.38 | 0.105 | 20 |
| Group 3 | 15.8 | 3.17 | N/A | N/A | N/A | N/A | 0.50 | 0.38 | 0.105 | 20 |
| Group 4 | 15.7 | 3.17 | N/A | N/A | N/A | N/A | 0.50 | 0.38 | 0.105 | 20 |
| Group 5 | 15.7 | 3.17 | N/A | N/A | N/A | N/A | 0.50 | 0.38 | 0.105 | 20 |
| Group 6 | 8.5 | N/A | 6.00 | 2.4 | 0.05 | 2.00 | 0.50 | 0.38 | 0.105 | 20 |
| Group 7 | 8.5 | N/A | 6.00 | 2.4 | 0.05 | 2.00 | 0.50 | 0.38 | 0.105 | 20 |
| Group 8 | 8.5 | N/A | 6.00 | 2.4 | 0.05 | 2.00 | 0.50 | 0.38 | 0.105 | 20 |

Table 8 shows the antigen content and recoveries for the three antigens. Control groups 9 and 10 formulated manually show good recovery for all antigens (90-110%), except for PE-PilA antigens. It was expected this was due to the initial concentration of these antigens (initial concentration was not verified before the experiment). For Protein D and PE-PilA, all continuous process groups showed high recoveries aligned with control groups 9 and 10 meaning there was no significant impact by the continuous process. Content and recovery results concerning UspA2 demonstrated variability. Low recovery was observed for groups 1 and 6, whereas high recovery was achieved for the other groups. Groups 1 and 6 involve addition of all antigens at the same time using the same micromixer. Using a specific micromixer per antigen seems to avoid this phenomenon.

TABLE 8

| | Protein D (ppm) | | PE-PilA | | UspA2 (ppm) | |
|---|---|---|---|---|---|---|
| | Measured | Recovery | Measured | Recovery | Measured | Recovery |
| Group | (ppm) | (%) | (ppm) | (%) | (ppm) | (%) |
| 1 | 20.05 | 89.51 | 25.07 | 111.9 | 4.95 | 66.89 |
| 2 | 21.02 | 93.84 | 25.21 | 112.5 | 6.74 | 91.08 |
| 3 | 21.83 | 97.46 | 25.52 | 113.9 | 5.96 | 80.54 |
| 4 | 21.64 | 96.61 | 25.69 | 114.7 | 7.03 | 95.00 |
| 5 | 20.68 | 92.32 | 24.85 | 110.9 | 7.07 | 95.54 |
| 6 | 20.53 | 91.65 | 24.79 | 110.7 | 4.42 | 59.73 |
| 7 | 21.82 | 97.41 | 25.72 | 114.8 | 5.71 | 77.16 |
| 8 | 22.32 | 99.64 | 25.74 | 114.9 | 6.67 | 90.14 |
| 9 | 21.93 | 97.90 | 25.19 | 112.5 | 6.93 | 93.65 |
| 10 | 21.13 | 94.33 | 24.69 | 110.2 | 7.07 | 95.54 |

The HPLC-SEC profiles were obtained for all groups to detect any antigen degradation. All were similar, with purity values in the expected range and similar to control groups 9 and 10. Small differences in terms of peak heights for UspA2 are observed for group 6 and are correlated with the lower antigen content measured with the UPLC content method. These profiles do not indicate any degradation from the continuous process and the use of micromixers, nor the micromixer setup.

### Example 5: Continuous Production of SAM-Containing LNPs

This experiment and the experiments performed in Examples 6 to 8 below utilised an apparatus comprising a set of pumps which sequentially introduced components to a stream of fluid. The components were delivered by centrifugal or gear pumps. Mixing was achieved using static micromixers connected in series. Process parameters were recoded using flow rate, pressure, weight and conductivity sensors.

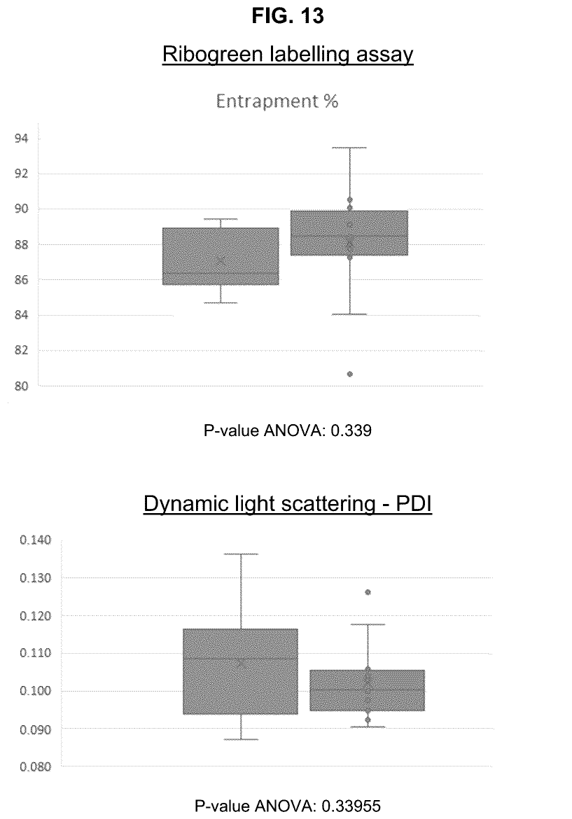

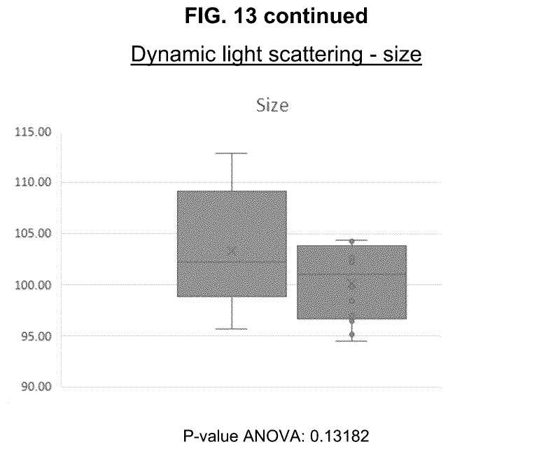

The continuous procedure of the invention was used for diluting SAM-containing LNP concentrate. The final formulation was compared to that produced by a classical batch process. Analyses were performed by Ribogreen Assay and DLS. A one variable ANOVA test was used to evaluate the statistical significance of the system as applicable. The results from the ANOVA test did not show any significant difference in terms of size, PDI or entrapment percentage (see FIG. 13, wherein bars on the left are from batch production and bars on the right are from continuous production according to the invention).

### Example 6: Continuous Shingles Vaccine Production

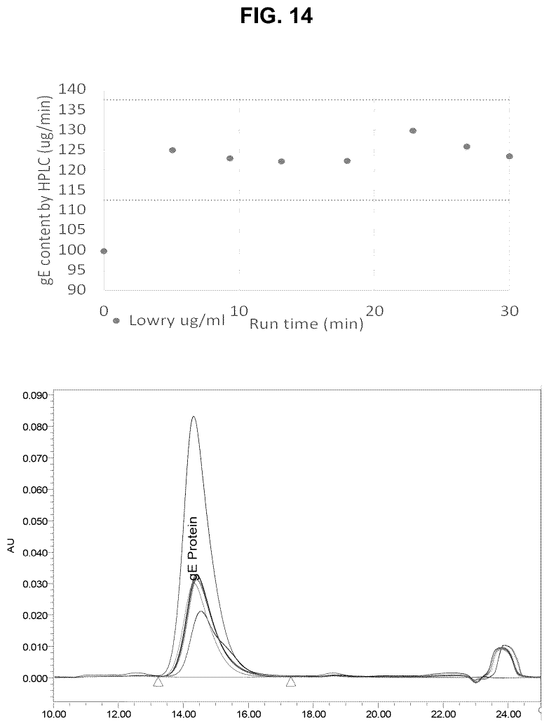

A shingles vaccine formulation comprising gE antigen was produced using the continuous production method of the invention. 3 litres of product were produced in 25 minutes. The product was analysed by HPLC SEC, Lowry and ELISA and the results compared to manufacturing criteria. Results are shown in Table 9 and FIG. 14.

TABLE 9

| Sample | Time | HPLC SEC | Lowry | |
|---|---|---|---|---|
| ID | min | ug/ml | ug/ml | ELISA |
| 1 | 0 | 102.74 | 99.7 | |
| 2 | 5 | 137.37 | 124.9 | 138 |
| 3 | 9 | 139.8 | 122.9 | 148 |
| 4 | 13 | 140.98 | 122.1 | 152 |
| 5 | 18 | 141.6 | 122.3 | 176 |
| 6 | 23 | 142.39 | 129.7 | 152 |
| 7 | 27 | 138.34 | 125.8 | 124 |
| 8 | 30 | 138.11 | 123.4 | 124 |

Product degradation was not observed in HPLC and SEC analyses. Content analysis by Lowry indicated stable product in samples collected sequentially and the ELISA assay showed acceptable potency results.

### Example 7: Continuous RSV Vaccine Production

An RSV vaccine formulation comprising RSV preF3 antigen was produced using the continuous production method of the invention. 10 litres of product were produced in 50 minutes. The product was analysed by ELISA for antigen purity. The purity by ELISA was found to have an acceptable level of variation between samples, as shown in Table 10 below.

TABLE 10

| | % RPD of PreF |
|---|---|
| Batch | Purity (%) |
| 1 | 92 |
| 2 | 102 |
| 3 | 94 |
| 4 | 106 |
| 5 | 87 |
| 6 | 104 |
| 7 | 84 |
| 8 | 90 |

### Example 8: Continuous Shingles Vaccine Production

Two batches were made and compared in terms of PFU/dose (plaque-forming units per dose). The first batch was prepared through continuous formulation. The second batch was prepared by the traditional batch method. Products of both batches presented PFU/dose differences but were acceptable in terms of quality. The results are shown in Tables 11 and 12.

TABLE 11

| | | | | Test | | | | |
|---|---|---|---|---|---|---|---|---|
| Titer as log | Pre-Test Dilution | | | Vial 1 | Vial 2 | | | |
| PFU/d | Dilution | Dilution | Dilution | dilution | dilution | | | Difference |
| (dose = 0.5 mL) | 1/20 | 1/40 | 1/100 | 1/40 | 1/40 | Avg. | SD | (2 − 1) |
| Continuous | 4.68 | 4.6 | 4.66 | 4.43 | 4.42 | 4.56 | 0.12 | |
| formulation | | | | | | | | |
| Batch | 4.77 | 4.77 | 4.87 | 4.64 | 4.79 | 4.77 | 0.08 | 0.21 |
| formulation | | | | | | | | |

TABLE 12
Continuous formulation kinetic - 1 vial (1/40)/timepoint

| T0 | T1 | T2 | T3 | T4 | T5 |
|---|---|---|---|---|---|
| 4.79 | 4.66 | 4.65 | 4.49 | 4.6 | 4.86 |

### REFERENCES

Haumont et al. Virus Research (1996) 40:199-204
Helminen et al. Infect. Immun. (1993) 61:2003-2010
Macher and Nickerson (2006) Pharmaceutical Manufacturing Research Project. Georgetown University McDonough School of Business and Washington University in St. Louis Olin School of Business
Vafai Vaccine (1994) 12:1265-1269
EP0405867
EP0192902
EP0281673
EP0594610
GB9917977.2
GB9918208.1
GB9918302.2
GB9918038.2
U.S. Pat. No. 5,766,608
U.S. Pat. No. 5,843,464
WO1991/018926
WO1993/003761
WO1994/012641
WO1994/026304
WO1996/034960
WO1997/001638
WO1997/013785
WO1997/032980
WO1997/041731
WO1998/055606
WO1999/057277
WO1999/058684
WO1999/063093
WO1999/064067
WO1999/064448
WO1999/064602
WO2000/015802
WO2000/052042
WO2005/063802
WO2006/094756
WO2007/084053
WO2012/139225
WO2015/125118
WO2021/023691
WO2021/023692

1. A continuous process for producing an immunogenic composition with a micro-fluidic or milli-fluidic (MF) system and filling one or more vessels with the immunogenic composition, the continuous process comprising:
a) introducing one or more antigens into the MF system,
b) introducing one or more further constituents into the MF system,
c) mixing the one or more constituents in the MF system thereby obtaining the immunogenic composition, and
d) removing the immunogenic composition from the MF system by filling the one or more vessels with the immunogenic composition.

2. The continuous process of claim 1, wherein the immunogenic composition is a vaccine.

3. The continuous process of claim 1, wherein the immunogenic composition is suitable for administration to a subject.

4. The continuous process of claim 1, wherein the one or more constituents are each independently one or more selected from carriers, buffers, isotonicity agents, stabilisers, bacteriostats, and cryoprotectants.

5. The continuous process of claim 1, wherein the mixing is performed with one or more micromixers.

6. The continuous process of claim 1, wherein the MF system comprises at least two inlets for introduction of the one or more constituents.

7. The continuous process of claim 1, wherein each of the one or more constituents is introduced by one pump per each of the one or more constituents.

8. The continuous process of claim 1, wherein the one or more vessels are tanks, bags, syringes, or vials.

9. The continuous process of claim 6, wherein each of the one or more constituents is introduced by a different inlet.

10. The continuous process of claim 1, wherein the immunogenic composition is sterile filtered after the mixing.

11. The continuous process of claim 1, wherein the one or more constituents are introduced at a higher concentration than a concentration at which they are present in the immunogenic composition.

12. The continuous process of claim 1, wherein a substantially homogenous immunogenic composition is produced.

13. The continuous process of claim 1, wherein the process does not adversely affect structure nor function of the one or more antigens.

14. The continuous process of claim 1, wherein less than 0.1 L of the immunogenic composition is wasted in priming the ME system before collecting.

15. The continuous process of claim 1, wherein less than 0.1 mL of the immunogenic composition is wasted per unit 10 L of the immunogenic composition produced.

16. The continuous process of claim 1, wherein less than 0.05 L of the immunogenic composition is wasted in priming the micro-fluidic or milli-fluidic system before collecting.

17. A micro-fluidic or milli-fluidic system for continuously producing an immunogenic composition and filling one or more vessels with the immunogenic composition, the micro-fluidic or milli-fluidic system comprising:
a) at least two inlets,
b) one or more mixers, and
c) one or more sterile filters.

18. A micro-fluidic or milli-fluidic system for producing an immunogenic composition, the micro-fluidic or milli-fluidic system comprising:
a) a line, which is for supplying a first fluid;
b) a first reservoir, which is for supplying a second fluid;
c) a second reservoir, which is for supplying a third fluid;
d) a first micromixer comprising:
i) a first inlet, which is in fluid communication with the line;
ii) a second inlet, which is in fluid communication with the first reservoir;
iii) a first outlet; and
iv) a first channel, which is for a first mixing; the first mixing being of the first fluid and the second fluid; the first channel comprising a first input and a second output; wherein:
A) the first inlet and the second inlet merge upstream of the first input;
B) the first fluid and the second fluid undergo the first mixing during passage through the first channel to form a first mixed fluid; and
C) the first output is in fluid communication with the first outlet; and
e) a second micromixer comprising:
i) a third inlet, which is in fluid communication with the first outlet,
ii) a fourth inlet, which is in fluid communication with the second reservoir;
ii) a second outlet; and
iii) a second channel, which is for a second mixing; the second mixing being of the first mixed fluid and a third fluid; the second channel comprising a second input and a second output; wherein:
A) the third inlet and the fourth inlet merge upstream of the second input;
B) the first mixed fluid and the third fluid undergo the second mixing during passage through the second channel to form a second mixed fluid; and
C) the second output is in fluid communication with the second outlet.

19. The micro-fluidic or milli-fluidic system of claim 17 further comprising elongate fluid flow channels, wherein:
a) the elongate fluid flow channels of the micro-fluidic system has an internal diameter not greater than 999 μm; and
b) the elongate fluid flow channels of the milli-fluidic system has an internal diameter from 1 mm to 100 mm.

20. The micro-fluidic or milli-fluidic system of claim 18, wherein:
a) the first channel and second channel of the micro-fluidic system each have an internal diameter not greater than 999 μm; and
b) the first channel and second channel of the milli-fluidic system each have an internal diameter from 1 mm to 100 mm.


SEQUENCE LISTING (4 sequences)

SEQ ID NO: 1 - protein, 696 aa, synthetic construct
  REGION 1..696: note = Artificial
MDPSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETKDGKQAQVYPNRFPLWKSHFRIHTFEDEIEFIQGLEKSTGKKVGIYPEIKAPWFHHQNGKDIAAETLKVLKKYGYDKKTDMVYLQTFDFNELKRIKTELLPQMGMDLKLVQLIAYTDWKETQEKDPKGYWVNYNYDWMFKPGAMAEVVKYADGVGPGWYMLVNKEESKPDNIVYTPLVKELAQYNVEVHPYTVRKDALPEFFTDVNQMYDALLNKSGATGVFTDFPDTGVEFLKGIKMDPSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETKDGKQAQVYPNRFPLWKSHFRIHTFEDEIEFIQGLEKSTGKKVGIYPEIKAPWFHHQNGKDIAAETLKVLKKYGYDKKTDMVYLQTFDFNELKRIKTELLPQMGMDLKLVQLIAYTDWKETQEKDPKGYWVNYNYDWMFKPGAMAEVVKYADGVGPGWYMLVNKEESKPDNIVYTPLVKELAQYNVEVHPYTVRKDALPEFFTDVNQMYDALLNKSGATGVFTDFPDTGVEFLKGIK

SEQ ID NO: 2 - protein, 275 aa, synthetic construct
  REGION 1..275: note = Artificial
MKYLLPTAAAGLLLLAAQPAMAIQKAEQNDVKLAPPTDVRSGYIRLVKNVNYYIDSESIWVDNQEPQIVHFDAVVNLDKGLYVYPEPKRYARSVRQYKILNCANYHLTQVRTDFYDEFWGQGLRAAPKKQKKHTLSLTPDTTLYNAAQIICANYGEAFSVDKKGGTKKAAVSELLQASAPYKADVELCVYSTNETTNCTGGKNGIAADITTAKGYVKSVTTSNGAITVKGDGTLANMEYILQATGNAATGVTWTTTCKGTDASLFPANFCGSVTQ

SEQ ID NO: 3 - protein, 537 aa, synthetic construct
  REGION 1..537: note = Artificial
MAKNDITLEDLPYLIKKIDQNELEADIGDITALEKYLALSQYGNILALEELNKALEELDEDVGWNQNDIANLEDDVETLTKNQNALAEQGEAIKEDLQGLADFVEGQEGKILQNETSIKKNTQRNLVNGFEIEKNKDAIAKNNESIEDLYDFGHEVAESIGEIHAHNEAQNETLKGLITNSIENTNNITKNKADIQALENNVVEELFNLSGRLIDQKADIDNNINNIYELAQQQDQHSSDIKTLKKNVEEGLLELSGHLIDQKTDIAQNQANIQDLATYNELQDQYAQKQTEAIDALNKASSENTQNIEDLAAYNELQDAYAKQQTEAIDALNKASSENTQNIEDLAAYNELQDAYAKQQTEAIDALNKASSENTQNIAKNQADIANNINNIYELAQQQDQHSSDIKTLAKASAANTDRIAKNKADADASFETLTKNQNTLIEKDKEHDKLITANKTAIDANKASADTKFAATADAITKNGNAITKNAKSITDLGTKVDGFDSRVTALDTKVNAFDGRITALDSKVENGMAAQAAHH

SEQ ID NO: 4 - protein, 516 aa, synthetic construct
  REGION 1..516: note = Artificial
SVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVNQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSYVDHFVNAIEERGFPPTAGQPPATTKPKEITPVNPGTSPLIRYAAWTGGLA